SEQUENCE LISTING (79 sequences)

SEQ ID NO: 1 - protein, 100 aa, Homo sapiens
RRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 2 - protein, 10 aa, Artificial sequence
  sequence is synthesized
KVASWTMFWV

SEQ ID NO: 3 - protein, 10 aa, Artificial sequence
  sequence is synthesized
WLDRFPHFWV

SEQ ID NO: 4 - protein, 9 aa, Artificial sequence
  sequence is synthesized
WEWIGMEWG

SEQ ID NO: 5 - protein, 9 aa, Artificial sequence
  sequence is synthesized
SHWWGGWLG

SEQ ID NO: 6 - protein, 9 aa, Artificial sequence
  sequence is synthesized
ATEFWWGVG

SEQ ID NO: 7 - protein, 9 aa, Artificial sequence
  sequence is synthesized
GIAGFWWDG

SEQ ID NO: 8 - protein, 9 aa, Artificial sequence
  sequence is synthesized
ESLWWGWEG

SEQ ID NO: 9 - protein, 9 aa, Artificial sequence
  sequence is synthesized
GGFWWGPAG

SEQ ID NO: 10 - protein, 9 aa, Artificial sequence
  sequence is synthesized
AGDSWWWGG

SEQ ID NO: 11 - protein, 7 aa, Artificial sequence
  sequence is synthesized
SWTMFWV

SEQ ID NO: 12 - protein, 7 aa, Artificial sequence
  sequence is synthesized
RFPHFWV

SEQ ID NO: 13 - protein, 7 aa, Artificial sequence
  sequence is synthesized
SHWWGGW

SEQ ID NO: 14 - protein, 6 aa, Artificial sequence
  sequence is synthesized
GQYYFV

SEQ ID NO: 15 - protein, 6 aa, Artificial sequence
  sequence is synthesized
GGIRRV

SEQ ID NO: 16 - protein, 8 aa, Artificial sequence
  sequence is synthesized
MDIELVMI

SEQ ID NO: 17 - protein, 6 aa, Artificial sequence
  sequence is synthesized
WTMFWV

SEQ ID NO: 18 - protein, 4 aa, Artificial sequence
  sequence is synthesized
SHWW

SEQ ID NO: 19 - protein, 15 aa, Artificial sequence
  sequence is synthesized
SMADPNRFRGKDLGS

SEQ ID NO: 20 - DNA, 69 bp, Artificial sequence
  sequence is synthesized
atcgacagcgcccccggtggcggannknnknnknnknnknnknnknnknnknnktgataaaccgataca

SEQ ID NO: 21 - DNA, 39 bp, Artificial sequence
  sequence is synthesized
ctgggcagcctcgagtaataataacgagaaccaaacttt

SEQ ID NO: 22 - DNA, 39 bp, Artificial sequence
  sequence is synthesized
gctgaactacagctttaataataagcacaccgggctggt

SEQ ID NO: 23 - DNA, 39 bp, Artificial sequence
  sequence is synthesized
ttggccattggggagtaataataacagatccggcgggga

SEQ ID NO: 24 - DNA, 93 bp, Artificial sequence
  sequence is synthesized
ctgggcagcctcgagsstsstkmtrytgstgytrygrygsytrctsytkccscakccrytsytgctgmasytsmasytcgagaaccaaacttt

SEQ ID NO: 25 - DNA, 96 bp, Artificial sequence
  sequence is synthesized
gctgaactacagcttsstgmascarmckytscagmtgytsmasmtgstgytsytrytsmtrmagytrytsytgstkccscagcacaccgggctggt

SEQ ID NO: 26 - DNA, 93 bp, Artificial sequence
  sequence is synthesized
ttggccattggggagsmaryggytsmarmcgctgmagmtgytkmtgmagctgytsstrctsmakccsmasytgcagytcagatccggcgggga

SEQ ID NO: 27 - protein, 10 aa, Artificial sequence
  sequence is synthesized
PGRWGPFFWV

SEQ ID NO: 28 - protein, 10 aa, Artificial sequence
  sequence is synthesized
DSLLFDFWWA

SEQ ID NO: 29 - protein, 10 aa, Artificial sequence
  sequence is synthesized
NQRVWIFWLI

SEQ ID NO: 30 - protein, 9 aa, Artificial sequence
  sequence is synthesized
SSFFRFWFV

SEQ ID NO: 31 - protein, 10 aa, Artificial sequence
  sequence is synthesized
DRLNWLFFWI

SEQ ID NO: 32 - protein, 10 aa, Artificial sequence
  sequence is synthesized
YPTYWTFWWV

SEQ ID NO: 33 - protein, 10 aa, Artificial sequence
  sequence is synthesized
LYSIYRFFWA

SEQ ID NO: 34 - protein, 10 aa, Artificial sequence
  sequence is synthesized
FLGFLEFFWI

SEQ ID NO: 35 - protein, 10 aa, Artificial sequence
  sequence is synthesized
SFYILRYFWV

SEQ ID NO: 36 - protein, 10 aa, Artificial sequence
  sequence is synthesized
TMSDWLFWWA

SEQ ID NO: 37 - protein, 10 aa, Artificial sequence
  sequence is synthesized
YGGTFILPHL

SEQ ID NO: 38 - protein, 10 aa, Artificial sequence
  sequence is synthesized
TRANWLFFWV

SEQ ID NO: 39 - protein, 10 aa, Artificial sequence
  sequence is synthesized
RIPFLFFLWA

SEQ ID NO: 40 - protein, 10 aa, Artificial sequence
  sequence is synthesized
SKLRLFFMWV

SEQ ID NO: 41 - protein, 10 aa, Artificial sequence
  sequence is synthesized
TGMSWTIWFL

SEQ ID NO: 42 - protein, 10 aa, Artificial sequence
  sequence is synthesized
SLLNWVLYLV

SEQ ID NO: 43 - protein, 10 aa, Artificial sequence
  sequence is synthesized
GLMPLLFFWV

SEQ ID NO: 44 - protein, 10 aa, Artificial sequence
  sequence is synthesized
TVHSWFLWFV

SEQ ID NO: 45 - protein, 10 aa, Artificial sequence
  sequence is synthesized
WVDSCPIFWV

SEQ ID NO: 46 - protein, 10 aa, Artificial sequence
  sequence is synthesized
IPLHWIFYLV

SEQ ID NO: 47 - protein, 7 aa, Artificial sequence
  sequence is synthesized
RWTIWFI

SEQ ID NO: 48 - protein, 7 aa, Artificial sequence
  sequence is synthesized
LWRFFWA

SEQ ID NO: 49 - protein, 9 aa, Artificial sequence
  sequence is synthesized
SHWWGGWLG

SEQ ID NO: 50 - protein, 9 aa, Artificial sequence
  sequence is synthesized
ATEFWWGVG

SEQ ID NO: 51 - protein, 9 aa, Artificial sequence
  sequence is synthesized
WGYWWGPGG

SEQ ID NO: 52 - protein, 9 aa, Artificial sequence
  sequence is synthesized
STDYWWGCG

SEQ ID NO: 53 - protein, 9 aa, Artificial sequence
  sequence is synthesized
GDIVCTWGG

SEQ ID NO: 54 - protein, 9 aa, Artificial sequence
  sequence is synthesized
SSDYWWGCG

SEQ ID NO: 55 - protein, 9 aa, Artificial sequence
  sequence is synthesized
GIVWFWWDG

SEQ ID NO: 56 - protein, 9 aa, Artificial sequence
  sequence is synthesized
WIAGFWWDG

SEQ ID NO: 57 - protein, 7 aa, Artificial sequence
  sequence is synthesized
SWTMFWV

SEQ ID NO: 58 - protein, 7 aa, Artificial sequence
  sequence is synthesized
SWTMFWA

SEQ ID NO: 59 - protein, 7 aa, Artificial sequence
  sequence is synthesized
SWTMFAV

SEQ ID NO: 60 - protein, 7 aa, Artificial sequence
  sequence is synthesized
SWTMAWV

SEQ ID NO: 61 - protein, 7 aa, Artificial sequence
  sequence is synthesized
SWTAFWV

SEQ ID NO: 62 - protein, 7 aa, Artificial sequence
  sequence is synthesized
SWAMFWV

SEQ ID NO: 63 - protein, 7 aa, Artificial sequence
  sequence is synthesized
SATMFWV

SEQ ID NO: 64 - protein, 7 aa, Artificial sequence
  sequence is synthesized
AWTMFWV

SEQ ID NO: 65 - protein, 5 aa, Artificial sequence
  sequence is synthesized
TMFWV

SEQ ID NO: 66 - protein, 4 aa, Artificial sequence
  sequence is synthesized
MFWV

SEQ ID NO: 67 - protein, 8 aa, Artificial sequence
  sequence is synthesized
SHWWGGWL

SEQ ID NO: 68 - protein, 6 aa, Artificial sequence
  sequence is synthesized
SHWWGG

SEQ ID NO: 69 - protein, 5 aa, Artificial sequence
  sequence is synthesized
SHWWG

SEQ ID NO: 70 - protein, 8 aa, Artificial sequence
  sequence is synthesized
HWWGGWLG

SEQ ID NO: 71 - protein, 7 aa, Artificial sequence
  sequence is synthesized
WWGGWLG

SEQ ID NO: 72 - protein, 6 aa, Artificial sequence
  sequence is synthesized
WGGWLG

SEQ ID NO: 73 - protein, 8 aa, Artificial sequence
  sequence is synthesized
SHWWGGAL

SEQ ID NO: 74 - protein, 8 aa, Artificial sequence
  sequence is synthesized
SHWAGGWL

SEQ ID NO: 75 - protein, 8 aa, Artificial sequence
  sequence is synthesized
SHAWGGWL

SEQ ID NO: 76 - protein, 8 aa, Artificial sequence
  sequence is synthesized
SAWWGGWL

SEQ ID NO: 77 - protein, 101 aa, Homo sapiens
KKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 78 - protein, 100 aa, Homo sapiens
KRFIGIRMRTITPSLVDELKASNPDFPEVSSGIYVQEVAPNSPSQRGGIQDGDIIVKVNGRPLVDSSELQEAVLTESPLLLEVRRGNDDLLFSIAPEVVM

SEQ ID NO: 79 - protein, 99 aa, Homo sapiens
KKYLGLQMLSLTVPLSEELKMHYPDFPDVSSGVYVCKVVEGTAAQSSGLRDHDVIVNINGKPITTTTDVVKALDSDSLSMAVLRGKDNLLLTVIPETIN

(19) United States
(12) Patent Application Publication
(10) Pub. No.: US 2009/0192289 A1
(43) Pub. Date: Jul. 30, 2009

(54) OMI PDZ MODULATORS

(75) Inventors: Sachdev S. Sidhu, Toronto (CA); Yingnan Zhang, Brisbane, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/270,517

(22) Filed: Nov. 13, 2008

Related U.S. Application Data
(62) Division of application No. 11/107,096, filed on Apr. 15, 2005, now abandoned.
(60) Provisional application No. 60/563,157, filed on Apr. 16, 2004.

(51) Int. Cl. C07K 2/00

(52) U.S. Cl. 530/300

(57) ABSTRACT

The invention provides modulators of Omi PDZ-ligand interaction, and methods of identifying and using these modulators.

### RELATED APPLICATION

This application is a divisional application claiming priority to U.S. application Ser. No. 11/107,096 filed Apr. 15, 2005 which claims benefit of U.S. Provisional Application No. 60/563,157 filed Apr. 16, 2004, the contents of which are incorporated herein in their entirety by reference for all purposes.

### BACKGROUND

Omi, also known as HtrA2, is a mammalian mitochondrial serine protease with extensive homology to bacterial high-temperature requirement A protease (HtrA)(1). Bacterial HtrA has a dual role acting as chaperone at normal temperatures and an active protease at elevated temperatures to dispose denatured or damaged proteins allowing the survival of bacteria following heat shock or other stress(2). Similar to HtrA in bacteria, the proteolytic activity of Omi is markedly up-regulated upon stress-activation(3).

The full-length Omi/HtrA2 protein contains 458 amino acids. The mature protein is produced by removal of 133 terminal residues. An IAP-binding motif, AVPS, is exposed by such processing(4-7). Omi/HtrA2 was originally identified as an IAP binding protein(4) and was believed to act as a promoter of apoptosis in mammalian cells via its ability to disrupt IAP-caspase interaction(4, 7-9). Studies indicate that this is not the only way through which Omi/HtrA2 induces apoptosis. It could induce cell death, either apoptosis(9-11) or necrosis(12), in a caspase-independent manner as well through its protease activity.

Data from mnd2 mutant mice have pointed to another function for Omi(13). Jones et. al. reported that mice with mutant Omi/HtrA2 suffer from a neurodegenerative disease. But rather than having extra cells, they found that loss of Omi protease activity causes progressive mitochondria damage. This suggests that one function of Omi/HtrA2 is to maintain mitochondria properly upon stress by handling misfolded proteins in the intermembrane space of mitochondria.

Mature Omi/HtrA2 contains a protease domain and a PDZ domain. The crystal structure of Omi/HtrA2 reveals that the PDZ domain packs against the protease domain with peptide-binding pocket of the PDZ domain buried in the intimate interface(16). The substrate access to protease catalytic site is therefore blocked by the PDZ domain. Disruption of PDZ/protease domain packing by mutating PDZ/protease interface(16) or engaging PDZ domain to peptide binding(11) can activate serine protease activity.

The carboxyl terminal sequence of Mxi2, a mitogen-activated protein kinase, has been suggested as an in vivo ligand for Omi PDZ by immunoprecipitation and yeast two hybrid assay(1). An optimized peptide ligand derived from a chemically synthesized peptide library that was void of tryptophan has also been reported(11).

The important cellular functions ascribed to Omi, in particular those mediated through the protein-protein interaction between Omi PDZ domain and ligand, suggest that Omi PDZ represents a significant therapeutic target. It would therefore be beneficial to elucidate the mechanistic aspects of Omi PDZ-ligand interaction and provide compositions and methods targeted at modulating its associated functional activities. The present invention provides this and other benefits.

### DISCLOSURE OF THE INVENTION

The present invention provides compositions, and methods of using these compositions, for modulating activity of the PDZ domain of the Omi protein. Because of the important functions associated with Omi, compositions and methods of the invention present significant clinical utilities. The invention is based in part on extensive analysis and characterization of binding partners (ligands) of Omi PDZ, said analysis resulting in novel and unexpected findings as described herein.

Two groups of peptide ligands to Omi PDZ were generated from phage-displayed libraries, with peptides fused either to the C-terminus or N-terminus of M13 p8 protein representing peptide binders that require a free carboxyl group and those that do not. Peptide ligands of Omi PDZ that comprise a free carboxyl terminus are herein described. These results demonstrate that, unlike ligands of most other PDZ domains that require having a free carboxyl terminus to be able to bind to PDZ, a subset of Omi PDZ ligands are capable of binding to Omi PDZ without a free carboxyl terminus. Ligands without a free carboxyl terminus represent N-terminus and/or internal Omi PDZ ligand sequences that are N-terminal or internal sequences of polypeptides.

As described below, binding specificities of a series of peptide ligands were assessed by measuring their relative affinities. Alanine scanning analysis was performed on the individual residues of an exemplary peptide ligand to elucidate the energetic contribution of different residues at each ligand position. Molecular modeling was also performed to dock an exemplary ligand to Omi PDZ domain to further assess the binding specificity on a structural basis. An efficient phage-based combinatorial scanning approach was also utilized to identify the residues in Omi PDZ domain that contribute energetically to ligand-PDZ interaction, providing further insight regarding structure and energetic components of Omi PDZ domain interaction with its ligands. As described in greater detail below, it is herein shown that ligands that interact with Omi PDZ domain comprise stretches of hydrophobic residues, either with free carboxyl terminus, or as N-terminal or internal polypeptide sequences (which is characteristic of denatured or damaged proteins in vivo).

In one aspect, the invention provides molecules capable of specifically binding Omi PDZ. These molecules are useful in a variety of contexts, for example as modulators of Omi PDZ-ligand interaction. For example, the invention provides modulator molecules having characteristics that mimic the characteristics of high, low or moderate affinity binders of Omi PDZ. In one embodiment, the invention provides an isolated polypeptide (e.g., a polypeptide as defined hereinbelow, which specifically includes peptide molecules) that binds specifically to Omi PDZ, wherein said polypeptide comprises a sequence having two hydrophobic moieties separated by 1, 2, 3, 4 or 5 amino acid positions. In one embodiment, at least one of the hydrophobic moieties is in the C-terminal region of the polypeptide. In one embodiment, one of the hydrophobic moieties comprises the carboxyl terminal amino acid residue of the polypeptide. In one embodiment, the two hydrophobic moieties are separated by at least one, two or three amino acid residues. In one embodiment, the two hydrophobic moieties are separated by about 1-5 amino acids, or about 1-4 amino acids, or about 1-3 amino acids, or about 2-5 amino acids, or about 2-4 amino acids, or about 3-5 amino acids, or about 3-4 amino acids, or about 4-5 amino acids. In one embodiment, one moiety comprises, consists of or consists essentially of about 2-4 hydrophobic clusters with aromatic residues in at least two amino acid positions and the other moiety comprises, consists of or consists essentially of about 1-2 hydrophobic amino acids with bulky side chain. In some embodiments, said other moiety comprises at least one amino acid with bulky side chain which is Trp, Phe, Leu or Ile or is selected from the group consisting of Trp, Phe, Leu and Ile.

In some contexts, the nature of the end terminal residue in a binder polypeptide can affect the binding capability of a polypeptide. Accordingly, in one embodiment, an isolated Omi PDZ-binding polypeptide of the invention comprises a carboxyl terminal amino acid residue which is carboxylated. In one embodiment, an isolated Omi PDZ-binding polypeptide of the invention comprises a carboxyl terminal amino acid residue that is missing a free carboxyl group.

Polypeptides of the invention can comprise specific amino acid residues in specific positions in the polypeptide sequence. In one embodiment, amino acid position −1 of a polypeptide of the invention is W, wherein amino acid numbering is based on the C-terminus residue being in position 0. In one embodiment, position −2 is F, wherein amino acid numbering is based on the C−terminus residue being in position 0. In one embodiment, position −3 is M, wherein amino acid numbering is based on the C−terminus residue being in position 0. In one embodiment, a first hydrophobic moiety comprises the amino acids FWV, wherein F is in position −2, W in position −1 and V in position 0, and wherein position 0 is the C-terminal residue. In one embodiment, a second hydrophobic moiety comprises T in position −4. In one embodiment, a second hydrophobic moiety comprises F in position −5. In one embodiment, a polypeptide comprises a combination of one or more of the positions listed above, wherein each of said positions comprises the corresponding amino acid as listed.

In one embodiment, the two hydrophobic moieties in a polypeptide of the invention has the formula X1-H1-X2-X3-H2-X4-X5, wherein H1 and H2 are a first and second hydrophobic moiety respectively. In one embodiment, X1 is the N-terminal residue. In one embodiment, X1 and X5 are not terminal residues. In one embodiment, H1 comprises a tripeptide sequence A1-A2-A3 and A1 is H. In one embodiment, H1 comprises a tripeptide sequence A1-A2-A3 and A2 is W. In one embodiment, H2 is W. In one embodiment, H1 comprises a tripeptide sequence A1-A2-A3 wherein A1 is H and A2 is W. In one embodiment, X1 is S.

In one embodiment, polypeptides of the invention specifically exclude Omi PDZ binder polypeptides that do not exhibit a desirable characteristic (such as binding affinity, e.g., wherein an example of a desirable characteristic is high affinity binding) of a binder peptide as disclosed herein (see, e.g., the Examples). For example, in one embodiment, a polypeptide of the invention does not comprise sequence GQYYFV (SEQ ID NO:14), GGIRRV (SEQ ID NO:15) or MDIELVMI (SEQ ID NO:16) wherein the C-terminal residue is carboxylated (i.e., if the sequence is in a polypeptide of the invention, the respective C-terminal residues, namely V, V and I, are not carboxylated or otherwise have a free carboxyl group). In another embodiment, a polypeptide of the invention does not comprise the sequence GQYYFV (SEQ ID NO:14), GGIRRV (SEQ ID NO:15) or MDIELVMI (SEQ ID NO:16).

In one aspect, the invention provides an isolated polypeptide that binds specifically to Omi PDZ and comprises either a carboxyl terminal, N-terminal or internal amino acid sequence having the sequence of a member selected from the group consisting of the sequences of Tables II and III. In one embodiment, the carboxyl terminal amino acid sequence has the sequence WTMFWV (SEQ ID NO:17). In one embodiment, the carboxyl terminal amino acid sequence has the sequence RFPHFWV (SEQ ID NO:12). In one aspect, the invention provides an isolated polypeptide that binds specifically to Omi PDZ and consists essentially of or consists of the sequence of a member selected from the group consisting of the sequences in Tables II and III. In one embodiment, the invention provides an isolated polypeptide that competes with any of the peptide in Tables II and III for binding to Omi PDZ sequence. In one embodiment, the invention provides an isolated polypeptide that binds to the same epitope on Omi PDZ as any of the peptide in Tables II and III. In one embodiment, an isolated polypeptide that competes with any of the peptide in Tables II and III for binding to Omi PDZ sequence does not comprise the sequence GQYYFV (SEQ ID NO: 14), GGIRRV (SEQ ID NO:15) or MDIELVMI (SEQ ID NO:16) wherein the C-terminal residue is carboxylated (i.e., if the sequence is in a polypeptide of the invention, the respective C-terminal residues, namely V, V and I, are not carboxylated or otherwise have a free carboxyl group). In another embodiment, an isolated polypeptide that competes with any of the peptide in Tables II and III for binding to Omi PDZ sequence does not comprise the sequence GQYYFV (SEQ ID NO:14), GGIRRV (SEQ ID NO:15) or MDIELVMI (SEQ ID NO:16).

In one aspect, the invention provides isolated polypeptides comprising an Omi PDZ variant sequence which is capable of interacting with an Omi PDZ ligand in vitro and/or in vivo. In one embodiment, an isolated polypeptide of the invention comprises, consists or consists essentially of an Omi PDZ variant sequence wherein Met232, Met233 and/or Tyr295 is substituted with another amino acid, wherein amino acid numbering corresponds to the numbering of human Omi protein, e.g. as described in the Examples. In one embodiment, an isolated polypeptide of the invention comprises, consists or consists essentially of an Omi PDZ variant sequence wherein His261 and/or Ile264 is substituted with another amino acid, wherein amino acid numbering corresponds to the numbering of human Omi protein, e.g. as described in the Examples. In one embodiment, said another amino acid is alanine. In one embodiment, the invention provides an isolated polypeptide that competes with an isolated polypeptide comprising, consisting or consisting essentially of an Omi PDZ variant sequence of the invention for binding to a ligand of Omi PDZ domain. In one embodiment, the invention provides an isolated polypeptide that binds to the same epitope on a ligand of Omi PDZ domain as an isolated polypeptide comprising, consisting or consisting essentially of an Omi PDZ variant sequence of the invention.

In one aspect, the invention provides useful methods for identifying compounds capable of modulating Omi PDZ-ligand interaction. These methods are obtained by utilizing Omi PDZ ligand characteristics and/or compositions described herein. For example, in one embodiment, the invention provides a method of identifying a compound capable of modulating Omi PDZ-ligand interaction, said method comprising contacting a sample comprising: (i) Omi PDZ, fragment thereof and/or a functional equivalent thereof; (ii) one or more of the Omi PDZ binding polypeptides of the invention (including any of the polypeptides described above, in particular the binding peptides of Tables II and Table III); and (iii) a candidate compound; and determining the amount of Omi PDZ-ligand interaction in the presence of the candidate compound; whereby a change in the amount of Omi PDZ-ligand interaction in the presence of the candidate compound compared to the amount in the absence of the compound indicates that the candidate compound is a compound capable of modulating Omi PDZ-ligand interaction. In another embodiment, the invention provides a method of rationally designing a modulator of Omi PDZ-ligand interaction comprising designing the modulator to comprise or mimic the function of two hydrophobic moieties separated by 1 or 2 amino acid position in a peptide, wherein the modulator is capable of specifically binding to Omi PDZ and/or modulating Omi PDZ-ligand interaction. In one embodiment of the method, the hydrophobic moieties are in the C-terminal region. In one embodiment of the method, one of the hydrophobic moieties of the peptide comprises the carboxyl terminal amino acid residue of the peptide. In one embodiment of the method, the two hydrophobic moieties of said peptide are separated by 1, 2, 3, 4 or 5 amino acid positions. In one embodiment, the two hydrophobic moieties are separated by at least one, two or three amino acid residues. In one embodiment, the two hydrophobic moieties are separated by about 1-5 amino acids, or about 1-4 amino acids, or about 1-3 amino acids, or about 2-5 amino acids, or about 2-4 amino acids, or about 3-5 amino acids, or about 3-4 amino acids, or about 4-5 amino acids. In one embodiment, the carboxyl terminal amino acid residue of said peptide is carboxylated. In one embodiment of the method, the amino acid at position −1 of said peptide is W, wherein amino acid numbering is based on the C-terminus residue being in position 0. In one embodiment of the method, the amino acid at position −2 of said peptide is F, wherein amino acid numbering is based on the C-terminus residue being in position 0. In one embodiment of the method, the amino acid at position −3 of said peptide is M, wherein amino acid numbering is based on the C-terminus residue being in position 0. In one embodiment of the method, a first hydrophobic moiety comprises the amino acids FWV, wherein F is in position −2, W in position −1 and V in position 0, and wherein position 0 is the C-terminal residue. In one embodiment of the method, a second hydrophobic moiety comprises T in position −4. In one embodiment of the method, a second hydrophobic moiety comprises F in position −5. In one embodiment of the method, the peptide sequence comprising two hydrophobic moieties has the formula X1-H1-X2-X3-H2-X4-X5, wherein H1 and H2 are a first and second hydrophobic moiety, respectively. In one embodiment, X1 is the N-terminal residue. In one embodiment, X1 and X5 are not terminal residues. In one embodiment, H1 comprises a sequence A1-A2-A3 and A1 is H. In one embodiment, H1 comprises a sequence A1-A2-A3 and A2 is W. In one embodiment, H1 comprises a sequence A1-A2-A3, and A1 is H and A2 is W. In one embodiment, H2 is W. In one embodiment, X1 is S.

Omi PDZ-ligand modulators of the invention are particularly useful in prophylactic, therapeutic and diagnostic methods targeted at pathological conditions associated with dysregulation of Omi protein activity, more specifically Omi PDZ-ligand interaction. Accordingly, in one aspect, the invention provides a method of treating a pathological condition (including any described herein) associated with dysregulation of Omi protein activity comprising administering to a subject in need thereof an effective amount of an Omi PDZ-ligand modulator, wherein the modulator is capable of modulating interaction between Omi PDZ and an Omi PDZ binding polypeptide of the invention (including any of the molecules described above). In one embodiment of the invention, said modulator inhibits interaction between Omi PDZ and said Omi PDZ binding polypeptide. In one embodiment of the invention, said modulator enhances interaction between Omi PDZ and said polypeptide. In one embodiment, the interaction that is modulated occurs in vivo, in vitro and/or ex vivo. Pathological conditions for which modulators of the invention are useful include those associated with dysregulation of cell death (e.g., caspase-dependent or caspase-independent) and/or improper protein quality control in mitochondria.

Modulator molecules of the invention can also be used for diagnostic purposes. Accordingly, in one aspect, the invention provides a method of identifying dysregulation of Omi PDZ-ligand interaction in a sample, said method comprising contacting the sample with a modulator molecule of the invention, and comparing Omi PDZ-ligand interaction in the presence and absence of the modulator whereby a detectable difference is indicative of the occurrence and/or amount of Omi PDZ-ligand interaction in the sample.

In another aspect, the invention provides a polynucleotide encoding a polypeptide of the invention (as described herein).

In another aspect, the invention provides a host cell comprising a polynucleotide and/or polypeptide of the invention (as described herein).

In another aspect, the invention provides a composition comprising one or more of the modulator molecules of the invention (as described herein). In one embodiment, the composition comprises a carrier, which in some embodiments is pharmaceutically acceptable.

In another aspect, the invention provides a kit comprising a comprising one or more of the modulator molecules of the invention (as described herein). When one or more modulator molecules are provided, they can be provided separately or together, so long as they are in a formulation suitable for an intended use. In one embodiment, the kit comprises instructions for using the composition.

### BRIEF DESCRIPTION OF THE DRAWINGS

FIG. 1 Sequence alignment of human HtrA family. The conserved residues are pasted dark grey; homologous conserved are light grey. Secondary structures are indicated with arrows and cylinders. The residues in Omi/HtrA2 that are scanned by shotgun libraries L1, L2 and L3 are underlined and labeled. Alanine mutations with F>16 are in bold and italic as well as labeled with *; those with (16>F>3.5) are labeled with +; those with F<0.3 are labeled with −.

### MODES FOR CARRYING OUT THE INVENTION

The invention provides molecules and methods for identifying and using molecules capable of modulating binding interactions between the PDZ domain of the Omi protein and its cellular binding partner(s). In one aspect, these molecules are generated by a combinatorial approach that results in the identification of peptide binders capable of binding to Omi PDZ at various affinities. The results described herein show that unexpectedly and significantly, Omi PDZ modulator molecules are capable of binding to Omi PDZ with or without a free carboxyl group. The identification of these binder molecules, and the structural dynamics of the binding interaction between Omi PDZ and its binding partners, as extensively described herein, further provide a means to identify other modulators capable of binding to Omi PDZ. In light of the importance of Omi in various cellular and physiological processes, these modulators would be of significant utility, such as in prophylactic, therapeutic and/or diagnostic settings.

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988).

Oligonucleotides, polynucleotides, peptides, polypeptides and small molecules employed or described in the present invention can be generated using standard techniques known in the art.

### DEFINITIONS

“Isolated,” when referred to a molecule, refers to a molecule that has been identified and separated and/or recovered from a component of its natural environment. Contaminant components of its natural environment are materials that interfere with diagnostic or therapeutic use.

“Control sequences”, as used herein, are DNA sequences that enable the expression of an operably-linked coding sequence in a particular host organism. Prokaryotic control sequences include promoters, operator sequences, and ribosome binding sites. Eukaryotic control sequences include promoters, polyadenylation signals, and enhancers.

Nucleic acid is “operably-linked” when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous.

An “active” polypeptide, or fragments thereof, retains a biological activity of native or naturally-occurring counterpart of the active polypeptide. Biological activity refers to a function mediated by the native or naturally-occurring counterpart of the active polypeptide. For example, binding or protein-protein interaction constitutes a biological activity.

The terms “antibody” and “immunoglobulin” are used interchangeably in the broadest sense and include monoclonal antibodies (e.g., full length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein).

The light chains of antibodies from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG-1, IgG-2, IgA-1, IgA-2, and etc. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

An antibody can be chimeric, human, humanized and/or affinity matured.

“Antibody fragments” comprise only a portion of an intact antibody, wherein the portion preferably retains at least one, preferably most or all, of the functions normally associated with that portion when present in an intact antibody.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “epitope tagged” polypeptide refers to a chimeric polypeptide fused to a “tag polypeptide”. Such tags provide epitopes against which Abs can be made or are available, but do not substantially interfere with polypeptide activity. To reduce anti-tag antibody reactivity with endogenous epitopes, the tag polypeptide is usually unique. Suitable tag polypeptides generally have at least six amino acid residues, usually between about 8 and 50 amino acid residues, preferably between 8 and 20 amino acid residues. Examples of epitope tag sequences include HA from Influenza A virus, GD, and c-myc, poly-His and FLAG.

“Polynucleotide,” or “nucleic acid,” as used interchangeably herein, refer to polymers of nucleotides of any length, and include, but are not limited to, DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR.sub.2 (“amidate”), P(O)R, P(O)OR’, CO or CH.sub.2 (“formacetal”), in which each R or R′ is independently H or substituted or unsubstituted alkyl (1-20° C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “peptide” generally refers to a contiguous and relatively short sequence of amino acids linked by peptidyl bonds. Typically, but not necessarily, a peptide has a length of about 2 to 50 amino acids, 4-40 amino acids or 10-30 amino acids. Although the term “polypeptide” generally refers to longer forms of a peptide, the two terms can be and are used interchangeably in some contexts herein.

A “region,” of a polypeptide is a contiguous sequence of 2 or more amino acids. In other embodiments, a region is at least about any of 3, 5, 10 contiguous amino acids. The “C-terminal region”, or variants thereof, refers to a region of a polypeptide that includes the 1-5 residues located closest to the C terminus of the polypeptide. The “N-terminal region”, or variants thereof, refers to a region of a polypeptide that includes the 1-5 residues located closest to the N terminus of the polypeptide.

A “PDZ domain”, which is also known as DHR (DLG homology region) or the GLGF repeat, is a protein domain originally described as conserved structural elements in the 95 kDa post-synaptic density protein (PSD-95), the Drosophila tumor suppressor discs-large, and the tight junction protein zonula occludens-1 (ZO-1), which are found in a large and diverse set of proteins, including the Omi protein. PDZ domains generally bind to short carboxyl-terminal peptide sequences located on the carboxyl-terminal end of interacting proteins. Usually, PDZ domains comprise two a helixes and six β sheets.

“Omi PDZ domain”, “OMI PDZ”, and variations thereof, refer to part or all of the sequence of SEQ ID NO:1, which is directly or indirectly involved in cellular Omi PDZ-ligand interactions.

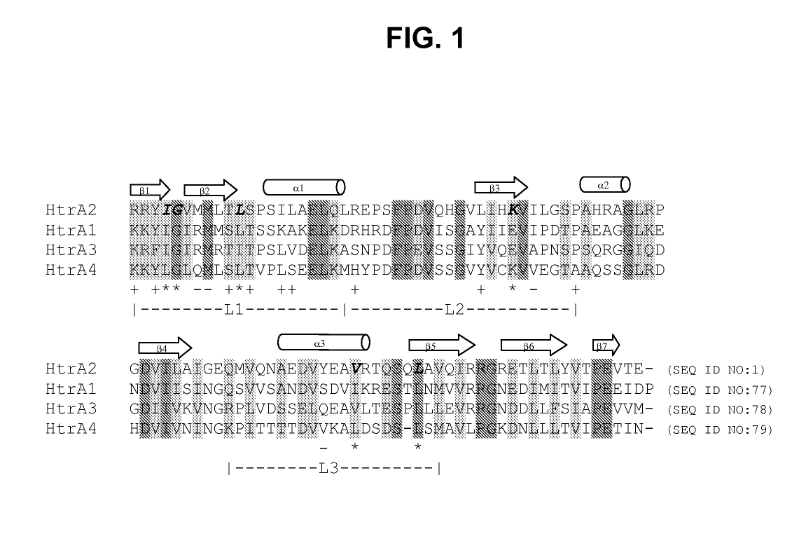

(SEQ ID NO:1; also see Figure 1)

RRYIGVMMLTLSPSILAELQLREPSFPDVQHGVLIHKVILGSPAHRAGLR
PGDVILAIGEQMVQNAEDVYEAVRTQSQLAVQIRRGRETLTLYVTPEVTE

A “ligand” refers to a naturally-occurring or synthetic molecule or moiety that is capable of a binding interaction with a specific site on a protein or other molecule; an Omi PDZ domain ligand is a molecule or moiety that specifically interactis with Omi PDZ domain. Examples of ligands include proteins, peptides, and small organic and inorganic molecules.

A “fusion protein” refers to a polypeptide having two portions covalently linked together, where each of the portions is derived from different proteins. The two portions may be linked directly by a single peptide bond or through a peptide linker containing one or more amino acid residues. Generally, the two portions and the linker will be in reading frame with each other and are produced using recombinant techniques.

A “disorder” or “pathological condition” is any condition that would benefit from treatment with a substance/molecule or method of the invention. This includes chronic and acute disorders or diseases including those pathological conditions which predispose the mammal to the disorder in question. Non-limiting examples of disorders to be treated herein include malignant and benign tumors or cancers; non-leukemias and lymphoid malignancies; neuronal, glial, astrocytal, hypothalamic and other glandular, macrophagal, epithelial, stromal and blastocoelic disorders; and inflammatory, immunologic, neurodegenerative disorders, angiogenesis-related disorders and disorders related to mitochondrial or metabolic defects.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, modulatory compounds of the invention are used to delay development of a disease or disorder.

An “effective amount” refers to an amount effective, at dosages and for periods of time necessary, to achieve the desired therapeutic or prophylactic result. A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

### Modulators of Omi PDZ-Ligand Interaction

The invention provides modulators, and methods for identifying modulators of Omi PDZ-ligand interaction in vivo. One way to modulate the interaction between Omi PDZ domain and its ligand is to inhibit the interaction. Any molecule that disrupts Omi PDZ-ligand interaction can be a candidate inhibitor. Screening techniques well known to those skilled in the art can identify these molecules. Examples of inhibitors include: (1) small organic and inorganic compounds, (2) small peptides, (3) antibodies and derivatives, (4) peptides closely related to PDZ-domain ligand (5) nucleic acid aptamers. “Omi PDZ-domain-ligand interaction inhibitor” includes any molecule that partially or fully blocks, inhibits, or neutralizes the interaction between Omi PDZ domain and its ligand. Molecules that may act as such inhibitors include peptides that bind Omi PDZ domain, such as the peptide binders listed in Tables II & III (for example and in particular peptides KVASWTMFWV (SEQ ID NO: 2); WLDRFPHFWV (SEQ ID NO:3); WEWIGMEWG (SEQ ID NO:4); SHWWGGWLG (SEQ ID NO:5); ATEFWWGVG (SEQ ID NO:6); GIAGFWWDG (SEQ ID NO:7); ESLWWGWEG (SEQ ID NO:8); GGFWWGPAG (SEQ ID NO:9); and AGDSWWWGG (SEQ ID NO:10); SWTMFWV (SEQ ID NO:11); RFPHFWV (SEQ ID NO:12); SHWWGGW [This is based on deletion description of libN binder on page 12 of disclosure] (SEQ ID NO:13)), antibodies (Ab's) or antibody fragments, fragments or variants of endogenous Omi PDZ domain ligands, cognate Omi PDZ-containing polypeptides; variants of Omi PDZ-containing polypeptides (e.g., wherein the Omi PDZ domain sequence comprises one or more substitutions at positions Met232, Met233, Tyr295, His261 and/or Ile264 (numbering according to human Omi protein amino acid sequence), for example substitution with an amino acid such as Ala or functional equivalent thereof), peptides, and small organic molecules.

### Small Molecule Omi PDZ Modulators

Small molecules can be useful modulators of Omi PDZ-ligand interaction. Small molecules that inhibit this interaction are potentially useful inhibitors. Examples of small molecule modulators include small peptides, peptide-like molecules, preferably soluble, and synthetic, non-peptidyl organic or inorganic compounds. A “small molecule” refers to a composition that has a molecular weight of preferably less than about 5 kD, preferably less than about 4 kD, and preferably less than 0.6 kD. Small molecules can be nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays. Examples of methods for the synthesis of molecular libraries have been described (Carell et al., Angewandte Chemie International Edition. 33:2059-2061 (1994); Carell et al., Angewandte Chemie International Edition. 33:2061-2064 (1994); Cho et al., Science. 261:1303-5 (1993); DeWitt et al., Proc Natl Acad Sci USA. 90:6909-13 (1993); Gallop et al., J Med. Chem. 37:1233-51 (1994); Zuckermann et al., J Med. Chem. 37:2678-85 (1994).

Libraries of compounds may be presented in solution (Houghten et al., Biotechniques. 13:412-21 (1992)) or on beads (Lam et al., Nature. 354:82-84 (1991)), on chips (Fodor et al., Nature. 364:555-6 (1993)), bacteria, spores (Ladner et al., U.S. Pat. No. 5,223,409, 1993), plasmids (Cull et al., Proc Natl Acad Sci USA. 89:1865-9 (1992)) or on phage (Cwirla et al., Proc Natl Acad Sci USA. 87:6378-82 (1990); Devlin et al., Science. 249:404-6 (1990); Felici et al., J Mol. Biol. 222:301-10 (1991); Ladner et al., U.S. Pat. No. 5,223,409, 1993; Scott and Smith, Science. 249:386-90 (1990)). A cell-free assay comprises contacting Omi PDZ with a known binder compound (such as one or more of the binder peptides described herein) to form an assay mixture, contacting the assay mixture with a test compound, and determining the ability of the test compound to interact with Omi PDZ or the binder compound, where determining the ability of the test compound to interact with Omi PDZ or the binder compound comprises determining whether a detectable characteristic of Omi PDZ/binder complex is modulated. For example, the binding interaction of Omi PDZ and the binder compound, as determined by the amount of complex that is formed, can be indicative of whether the test compound is able to modulate the interaction between Omi PDZ and the binder compound. Amount of complex can be assessed by methods known in the art, some of which are described herein, for example ELISA (including competitive binding ELISA), yeast two-hybrid and proximity (e.g., fluorescent resonance energy transfer, enzyme-substrate) assays.

### Polypeptide/Peptide and Antibody Omi PDZ Modulators

One aspect of the invention pertains to isolated peptide/polypeptide modulators of the interaction between Omi PDZ and its cellular and/or physiological binding partner(s). The binder peptides described herein, and peptide modulators obtained by methods described herein are also suitable for use as immunogens to raise antibody modulators of this interaction. In one embodiment, modulators (such as peptides and antibodies) can be isolated from cells or tissue sources by an appropriate purification scheme using standard protein purification techniques. In another embodiment, the modulators are produced by recombinant DNA techniques. Alternative to recombinant expression, modulators can be synthesized chemically using standard peptide synthesis techniques.

Omi PDZ binder peptides of the invention include those described in Tables II and III. The invention also provides a mutant or variant protein any of which residues may be changed from the corresponding residues of these peptides, while still encoding a peptide that maintains modulatory activity. In one embodiment, a variant of a binder peptide/polypeptide/ligand has at least 50%, 60%, 70%, 80%, 90%, 95%, 98%, 99% amino acid sequence identity with the sequence of a reference binder peptide/polypeptide/ligand. In general, the variant exhibits substantially the same or greater binding affinity than the reference binder peptide/polypeptide/ligand, e.g., at least 0.75×, 0.8×, 0.9×, 1.0×, 1.25× or 1.5× the binding affinity of the reference binder peptide/polypeptide/ligand, based on an art-accepted binding assay quantitation unit/metric.

In general, variants of the invention include variants in which residues at a particular position in the sequence have been substituted by other amino acids, and further includes the possibility of inserting an additional residue or residues between two residues of the parent protein/peptide as well as the possibility of deleting one or more residues from the parent sequence or adding one or more residues to the parent sequence. Any amino acid substitution, insertion, or deletion is encompassed by the invention. In favorable circumstances, the substitution is a conservative substitution as described herein.

“Percent (%) amino acid sequence identity” is defined as the percentage of amino acid residues that are identical with amino acid residues in a reference (parent) polypeptide sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B

and

Y is the total number of amino acid residues in B.

If the length of amino acid sequence A is not equal to the length of amino acid sequence B, the % amino acid sequence identity of A to B will not equal the % amino acid sequence identity of B to A.

An “isolated” or “purified” peptide, polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preparations having preferably less than 30% by dry weight of non-desired contaminating material (contaminants), preferably less than 20%, 10%, and preferably less than 5% contaminants are considered to be substantially isolated. An isolated, recombinantly-produced peptide/polypeptide or biologically active portion thereof is preferably substantially free of culture medium, i.e., culture medium represents preferably less than 20%, preferably less than about 10%, and preferably less than about 5% of the volume of a peptide/polypeptide preparation. Examples of contaminants include cell debris, culture media, and substances used and produced during in vitro synthesis of the peptide/polypeptide.

Conservative substitutions of peptides/polypeptides are shown in Table V under the heading of “preferred substitutions”. If such substitutions result in a change in biological activity, then more substantial changes, denominated “exemplary substitutions” in Table V, or as further described below in reference to amino acid classes, may be introduced and the products screened.

TABLE V

| | | Preferred |
|---|---|---|
| Original Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr; cys | cys |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the peptide/polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchanging a member of one of these classes for another class.

Variants of antibody modulators of Omi PDZ-ligand interaction can also be made based on information known in the art, without substantially affecting the activity of antibody. For example, antibody variants can have at least one amino acid residue in the antibody molecule replaced by a different residue. For antibodies, the sites of greatest interest for substitutional mutagenesis generally include the hypervariable regions, but framework region (FR) alterations are also contemplated.

For antibodies, one type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of the immunoglobulin polypeptides of the invention, thereby generating a Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In one embodiment, the Fc region variant may display altered neonatal Fc receptor (FcRn) binding affinity. Such variant Fc regions may comprise an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 382, 386, 388, 400, 413, 415, 424, 433, 434, 435, 436, 439 or 447 of the Fc region, wherein the numbering of the residues in the Fc region is that of the EU index as in Kabat. Fc region variants with reduced binding to an FcRn may comprise an amino acid modification at any one or more of amino acid positions 252, 253, 254, 255, 288, 309, 386, 388, 400, 415, 433, 435, 436, 439 or 447 of the Fc region, wherein the numbering of the residues in the Fc region is that of the EU index as in Kabat. The above-mentioned Fc region variants may, alternatively, display increased binding to FcRn and comprise an amino acid modification at any one or more of amino acid positions 238, 256, 265, 272, 286, 303, 305, 307, 311, 312, 317, 340, 356, 360, 362, 376, 378, 380, 382, 413, 424 or 434 of the Fc region, wherein the numbering of the residues in the Fc region is that of the EU index as in Kabat.

The Fc region variant with reduced binding to an FcγR may comprise an amino acid modification at any one or more of amino acid positions 238, 239, 248, 249, 252, 254, 265, 268, 269, 270, 272, 278, 289, 292, 293, 294, 295, 296, 298, 301, 303, 322, 324, 327, 329, 333, 335, 338, 340, 373, 376, 382, 388, 389, 414, 416, 419, 434, 435, 437, 438 or 439 of the Fc region, wherein the numbering of the residues in the Fc region is that of the EU index as in Kabat.

For example, the Fc region variant may display reduced binding to an FcγRI and comprise an amino acid modification at any one or more of amino acid positions 238, 265, 269, 270, 327 or 329 of the Fc region, wherein the numbering of the residues in the Fc region is that of the EU index as in Kabat.

The Fc region variant may display reduced binding to an FcγRII and comprise an amino acid modification at any one or more of amino acid positions 238, 265, 269, 270, 292, 294, 295, 298, 303, 324, 327, 329, 333, 335, 338, 373, 376, 414, 416, 419, 435, 438 or 439 of the Fc region, wherein the numbering of the residues in the Fc region is that of the EU index as in Kabat.

The Fc region variant of interest may display reduced binding to an FcγRIII and comprise an amino acid modification at one or more of amino acid positions 238, 239, 248, 249, 252, 254, 265, 268, 269, 270, 272, 278, 289, 293, 294, 295, 296, 301, 303, 322, 327, 329, 338, 340, 373, 376, 382, 388, 389, 416, 434, 435 or 437 of the Fc region, wherein the numbering of the residues in the Fc region is that of the EU index as in Kabat.

Fc region variants with altered (i.e. improved or diminished) C1q binding and/or Complement Dependent Cytotoxicity (CDC) are described in WO99/51642. Such variants may comprise an amino acid substitution at one or more of amino acid positions 270, 322, 326, 327, 329, 331, 333 or 334 of the Fc region. See, also, Duncan & Winter Nature 322:738-40 (1988); U.S. Pat. No. 5,648,260; U.S. Pat. No. 5,624,821; and WO94/29351 concerning Fc region variants.

### Vector Construction

Polynucleotide sequences encoding the peptide and polypeptides described herein can be obtained using standard synthetic and/or recombinant techniques. Desired polynucleotide sequences may be isolated and sequenced from appropriate source cells. Source cells for antibodies would include antibody producing cells such as hybridoma cells. Alternatively, polynucleotides can be synthesized using nucleotide synthesizer or PCR techniques. Once obtained, sequences encoding the peptide or polypeptide are inserted into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication (in particular when the vector is inserted into a prokaryotic cell), a selection marker gene, a promoter, a ribosome binding site (RBS), a signal sequence, the heterologous nucleic acid insert and a transcription termination sequence.

In general, plasmid vectors containing replicon and control sequences which are derived from a species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

Either constitutive or inducible promoters can be used in the present invention, in accordance with the needs of a particular situation, which can be ascertained by one skilled in the art. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of the target genes. However, heterologous promoters are preferred, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In some embodiments, each cistron within a recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

Prokaryotic host cells suitable for expressing polypeptides include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. Preferably, gram-negative cells are used. Preferably the host cell should secrete minimal amounts of proteolytic enzymes, and additional protease inhibitors may desirably be incorporated in the cell culture.

### Peptide or Polypeptide Production

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In preferred embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. For example, if a PhoA promoter is used for controlling transcription, the transformed host cells may be cultured in a phosphate-limiting medium for induction. A variety of other inducers may be used, according to the vector construct employed, as is known in the art.

Polypeptides described herein expressed in a microorganism may be secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therefrom. Cells may be removed from the culture and the culture supernatant being filtered and concentrated for further purification of the proteins produced. The expressed polypeptides can be further isolated and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable antigen immobilized on a matrix and Western blot assay.

Besides prokaryotic host cells, eukaryotic host cell systems are also well established in the art. Suitable hosts include mammalian cell lines such as CHO, and insect cells such as those described below.

### Polypeptide/Peptide Purification

Polypeptides/peptides that are produced may be purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

### Identification and Characterization of Omi PDZ Modulators—General Approach

Candidate Omi PDZ modulators, e.g. binding peptides, can be identified by any number of methods known in the art. The modulatory characteristics of modulators can be assessed by determining the ability of the modulators to modulate the interaction between Omi PDZ and its cellular binding partners. One of the important characteristics is binding affinity. The binding characteristics of candidate modulators (e.g. peptides) of interest can be assessed in any of a number of ways known in the art.

An initial step in the process can include generating one or more candidate peptides comprising sequences of interest, which are then displayed under conditions suitable to determine their Omi PDZ domain binding characteristics. For example, candidate peptides can be displayed as carboxyl-terminal (C-terminal) display libraries of peptides on the surface of a phage or phagemid, for example a filamentous phage(mid) using protein fusions with a coat protein such as p3 or p8. C-terminal display is known in the art. See, e.g., Jespers et al., Biotechnology (NY). 13:378-82 and WO 00/06717. These methods may be used to prepare the fusion genes, fusion proteins, vectors, recombinant phage particles, host cells and libraries thereof of the invention. As described herein, in some embodiments, it may be useful to display candidate peptides as amino-terminal (N-terminal) display libraries of peptides on the surface of a phage or phagemid. Methods of N-terminal phage(mid) display include those described herein, and those that are well known in the art, e.g., as described in U.S. Pat. No. 5,750,373 (and references cited therein). Methods of characterizing binder molecules obtained by these methods are also known in the art, including those disclosed in the references cited above (Jespers et al., WO 00/06717 & U.S. Pat. No. 5,750,373) and as described herein.

### (i) Isolation of Binding Phage to Omi PDZ

A phage display library with the displayed candidate Omi PDZ binding peptides is contacted with Omi PDZ domain proteins or fusion proteins in vitro to determine those members of the library that bind to an Omi PDZ domain target. Any method, known to the skilled artisan, may be used to assay for in vitro protein binding. For example, 1, 2, 3 or 4 rounds or more of binding selection may be performed, after which individual phage are isolated and, optionally, analyzed in a phage ELISA. Binding affinities of peptide-displaying phage particles to immobilized PDZ target proteins may be determined using a phage ELISA (Barrett et al., Anal Biochem. 204:357-64 (1992)).

In a situation wherein the candidate is being assessed for the ability to compete with a known Omi PDZ binder for binding to Omi PDZ, the appropriate binding competition conditions are provided. For example, in one embodiment, screening/selection/biopanning can be performed in the presence of one or more concentrations of the known Omi PDZ binder. In another embodiment, candidate binders isolated from the library can be subsequently assessed in a competitive ELISA assay in the presence of the known Omi PDZ binder.

### (ii) Preparation of Omi PDZ Domains

Omi PDZ domains may be produced conveniently as protein fragments containing the domain or as fusion polypeptides using conventional synthetic or recombinant techniques. Fusion polypeptides are useful in phage(mid) display wherein Omi PDZ domain is the target antigen, in expression studies, cell-localization, bioassays, ELISAs (including binding competition assays), etc. An Omi PDZ domain “chimeric protein” or “fusion protein” comprises Omi PDZ domain fused to a non-PDZ domain polypeptide. A non-PDZ domain polypeptide is not substantially homologous to the PDZ domain. An Omi PDZ domain fusion protein may include any portion to the entire PDZ domain, including any number of the biologically active portions. The fusion protein can then be purified according to known methods using affinity chromatography and a capture reagent that binds to the non-PDZ domain polypeptide. Omi PDZ domain may be fused to an affinity sequence, e.g. the C-terminus of the GST (glutathione S-transferase) sequences. Such fusion proteins facilitate the purification of the recombinant Omi PDZ domain using, e.g., glutathione bound to a solid support and/or attachment to solid support (e.g., a matrix for peptide screening/selection/biopanning). Additional exemplary fusions are presented in Table VI, including some common uses for such fusions.

Fusion proteins can be easily created using recombinant methods. A nucleic acid encoding Omi PDZ domain (or portion thereof) can be fused in-frame with a non-PDZ domain encoding nucleic acid, at the PDZ domain N-terminus, C-terminus or internally. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., Current protocols in molecular biology. John Wiley & Sons, New York 1987) is also useful. Many vectors are commercially available that facilitate sub-cloning the Omi PDZ domain in-frame to a fusion protein.

TABLE VI
Useful non-PDZ domain fusion polypeptides

| Fusion partner | in vitro | in vivo |
|---|---|---|
| Human growth | Radioimmuno-assay | none |
| hormone (hGH) | | |
| β-glucuronidase | Colorimetric, | colorimetric (histo- |
| (GUS) | fluorescent, or chemi- | chemical staining |
| | luminescent | with X-gluc) |
| Green fluorescent | Fluorescent | fluorescent |
| protein (GFP) and | | |
| related molecules | | |
| (RFP, BFP, YFP | | |
| domain, etc.) | | |
| Luciferase (firefly) | bioluminsecent | Bioluminescent |
| Chloramphenicoal | Chromatography, | none |
| acetyltransferase | differential extraction, | |
| (CAT) | fluorescent, or | |
| | immunoassay | |
| β-galacto-sidase | colorimetric, | colorimetric |
| | fluorescence, chemi- | (histochemical |
| | luminscence | staining with X-gal), |
| | | bio-luminescent in |
| | | live cells |
| Secrete alkaline | colorimetric, | none |
| phosphatase | bioluminescent, | |
| (SEAP) | chemi-luminescent | |
| Tat from HIV | Mediates delivery into | Mediates delivery |
| | cytoplasm and nuclei | into cytoplasm and |
| | | nuclei |

As an example of an Omi PDZ domain fusion, GST-Omi PDZ fusion may be prepared from a gene of interest in the following manner. With the full-length gene of interest as the template, the PCR is used to amplify DNA fragments encoding the PDZ domain using primers that introduce convenient restriction endonuclease sites to facilitate sub-cloning. Each amplified fragment is digested with the appropriate restriction enzymes and cloned into a similarly digested plasmid, such as pGEX6P-3 or pGEX-4T-3, that contains GST and is designed such that the sub-cloned fragments will be in-frame with the GST and operably linked to a promoter, resulting in plasmids encoding GST-Omi PDZ fusion proteins.

To produce the fusion protein, E. coli cultures harboring the appropriate expression plasmids are generally grown to mid-log phase (A₆₀₀=1.0) in LB broth, e.g. at about 37° C., and may be induced with IPTG. The bacteria are pelleted by centrifugation, resuspended in PBS and lysed by sonication. The suspension is centrifuged, and GST-Omi PDZ fusion proteins are purified from the supernatant by affinity chromatography on 0.5 ml of glutathione-Sepharose.

It will be apparent to one of skill in the art that many variations will achieve the goal of isolated Omi PDZ domain protein and may be used in this invention. For example, fusions of the Omi PDZ domain and an epitope tag may be constructed as described above and the tags used to affinity purify the Omi PDZ domain. Omi PDZ domain proteins/peptides may also be prepared without any fusions; in addition, instead of using the microbial vectors to produce the protein, in vitro chemical synthesis may instead be used. Other cells may be used to produce Omi PDZ domain proteins/peptides, such as other bacteria, mammalian cells (such as COS), or baculoviral systems. A wide variety of polynucleotide vectors to produce a variety of fusions are also available. The final purification of an Omi PDZ domain fusion protein will generally depend on the fusion partner; for example, a poly-histidine tag fusion can be purified on nickel columns.

(iii) Determining the Sequence of the Displayed Peptide

Phage(mid) that bind to Omi PDZ with the desired characteristics (and optionally, does not bind to unrelated sequences), can be subjected to sequence analysis. The phage(mid) particles displaying the candidate binding peptides are amplified in host cells, the DNA isolated, and the appropriate portion of the genome (encoding the candidate peptide) sequenced using any appropriate known sequencing technique.

### Other Approaches for Identifying Modulators of Omi PDZ-Ligand Interaction

Another approach to identify modulators of Omi PDZ-ligand binding is to incorporate rational drug design; that is, to understand and exploit the biology of the PDZ interaction. In this approach, the critical residues in a PDZ ligand are determined, as is, optionally, the optimal peptide length. Then, small molecules are designed with this information in hand; for example, if a tyrosine is found to be a critical residue for binding to a PDZ domain, then small molecules that contain a tyrosine residue will be prepared and tested as inhibitors. Generally 2, 3, 4 or 5 amino acid residues will be determined to be critical for binding and candidate small molecule inhibitors will be prepared containing these residues or the residue sidechains. The test compounds are then screened for their ability to inhibit Omi PDZ domain-ligand interactions using protocols well-known in the art, for example, a competitive inhibition assay.

Compounds that modulate Omi PDZ domain-ligand binding interactions are useful to treat diseases and conditions that are associated with dysregulation of binding interactions of Omi PDZ. Diseases and conditions that are associated with regulation of Omi PDZ domain interactions include caspase dependent and independent apoptosis, and mitochondria protein quality control.

1. Determining Critical Residues in an Omi PDZ Binding Polypeptide

(a) Alanine Scanning

Alanine scanning an Omi PDZ domain binding peptide sequence can be used to determine the relative contribution of each residue in the ligand to PDZ binding. To determine the critical residues in a PDZ ligand, residues are substituted with a single amino acid, typically an alanine residue, and the effect on PDZ domain binding is assessed. See U.S. Pat. No. 5,580,723; U.S. Pat. No. 5,834,250; and the Examples.

(b) Truncations (Deletion Series)

Truncation of an Omi PDZ domain binding peptide can elucidate not only binding critical residues, but also determine the minimal length of peptide to achieve binding. In some cases, truncation will reveal a ligand that binds more tightly than the native ligand; such a peptide is useful to modulate Omi PDZ domain:PDZ ligand interactions.

Preferably, a series of Omi PDZ-domain binding peptide truncations are prepared. One series will truncate the amino terminal amino acids sequentially; in another series, the truncations will begin at the carboxy terminus. As in the case for alanine scanning, the peptides may be synthesized in vitro or prepared by recombinant methods.

(c) Rational Modulator Design

Based on the information obtained from alanine scanning and truncation analysis, the skilled artisan can design and synthesize small molecules, or select small molecule libraries that are enriched in compounds that are likely to modulate binding. For example, based on the information as described in the Examples, a modulator peptide can be designed to include 2 appropriate-spaced hydrophobic moieties.

(d) Binding Assays

Forming a complex of an Omi PDZ binding peptide and Omi PDZ facilitates separation of the complexed from the uncomplexed forms thereof and from impurities. Omi PDZ domain:binding ligand complexes can be formed in solution or where one of the binding partners is bound to an insoluble support. The complex can be separated from a solution, for example using column chromatography, and can be separated while bound to a solid support by filtration, centrifugation, etc. using well-known techniques. Binding the PDZ domain containing polypeptide or the ligand therefor to a solid support facilitates high throughput assays.

Test compounds can be screened for the ability to modulate (e.g., inhibit) the interaction of a binder polypeptide with Omi PDZ domain in the presence and absence of a candidate binding compound, and screening can be accomplished in any suitable vessel, such as microtiter plates, test tubes, and microcentrifuge tubes. Fusion proteins can also be prepared to facilitate testing or separation, where the fusion protein contains an additional domain that allows one or both of the proteins to be bound to a matrix. For example, GST-PDZ-binding peptide fusion proteins or GST-PDZ domain fusion proteins can be adsorbed onto glutathione sepharose beads (SIGMA Chemical, St. Louis, Mo.) or glutathione derivatized microtiter plates that are then combined with the test compound or the test compound and either the nonadsorbed Omi PDZ domain protein or PDZ-binding peptide, and the mixture is incubated under conditions allowing complex formation (e.g., at physiological conditions of salt and pH). Following incubation, the beads or microtiter plate wells are washed to remove any unbound components, the matrix immobilized in the case of beads, and the complex determined either directly or indirectly. Alternatively, the complexes can be dissociated from the matrix, and the level of binding or activity determined using standard techniques.

Other fusion polypeptide techniques for immobilizing proteins on matrices can also be used in screening assays. Either an Omi PDZ binding peptide or Omi PDZ can be immobilized using biotin-avidin or biotin-streptavidin systems. Biotinylation can be accomplished using many reagents, such as biotin-N-hydroxy-succinimide (NHS; PIERCE Chemicals, Rockford, Ill.), and immobilized in wells of streptavidin coated 96 well plates (PIERCE Chemical). Alternatively, antibodies reactive with Omi PDZ binding peptides or Omi PDZ domain but do not interfere with binding of a binding peptide to its target molecule can be derivatized to the wells of the plate, and unbound Omi PDZ or binder peptide trapped in the wells by antibody conjugation. Methods for detecting such complexes, in addition to those described for the GST-immobilized complexes, include immunodetection of complexes using antibodies reactive with the binder peptides or Omi PDZ domain.

(e) Assay for Binding: Competition ELISA

To assess the binding affinities of a peptide, proteins or other Omi PDZ ligands, competition binding assays may be used, where the ability of the ligand to bind Omi PDZ domain (and the binding affinity, if desired) is assessed and compared to that of a compound known to bind the PDZ domain, for example, a high-affinity binder peptide determined by phage display as described herein.

Many methods are known and can be used to identify the binding affinities of binding molecules (e.g. peptides, proteins, small molecules, etc.); for example, binding affinities can be determined as IC₅₀ values using competition ELISAs. The IC₅₀ value is defined as the concentration of binder which blocks 50% of Omi PDZ domain binding to a ligand. For example, in solid phase assays, assay plates may be prepared by coating microwell plates (preferably treated to efficiently adsorb protein) with neutravidin, avidin or streptavidin. Non-specific binding sites are then blocked through addition of a solution of bovine serum albumin (BSA) or other proteins (for example, nonfat milk) and then washed, preferably with a buffer containing a detergent, such as Tween-20. A biotinylated known Omi PDZ binder (for example, the phage peptides as fusions with GST or other such molecule to facilitate purification and detection) is prepared and bound to the plate. Serial dilutions of the molecule to be tested with Omi PDZ domain are prepared and contacted with the bound binder. The plate coated with the immobilized binder is washed before adding each binding reaction to the wells and briefly incubated. After further washing, the binding reactions are detected, often with an antibody recognizing the non-PDZ fusion partner and a labeled (such as horseradish peroxidase (HRP), alkaline phosphatase (AP), or a fluorescent tag such as fluorescein) secondary antibody recognizing the primary antibody. The plates are then developed with the appropriate substrate (depending on the label) and the signal quantified, such as using a spectrophotometric plate reader. The absorption signal may be fit to a binding curve using a least squares fit. Thus the ability of the various molecules to inhibit PDZ domain from binding a known PDZ domain binder can be measured.

Apparent to one of skill are the many variations of the above assay. For example, instead of avidin-biotin based systems, PDZ domain binders may be chemically-linked to a substrate, or simply adsorbed.

2. PDZ Domain Peptide Ligands Found During Phage Display

PDZ domain peptide ligands, even those that bind with relatively lower affinity (e.g., compared to a consensus sequence), are potential useful inhibitors of the Omi PDZ-ligand interaction, including those described in the Examples (and Tables II and III).

The competitive binding ELISA is a useful means to determine the efficacy of each phage-displayed PDZ-domain binding peptide.

3. Aptamers

Aptamers are short oligonucleotide sequences that can be used to recognize and specifically bind almost any molecule. The systematic evolution of ligands by exponential enrichment (SELEX) process (Ausubel et al., Current protocols in molecular biology. John Wiley & Sons, New York (1987); Ellington and Szostak, Nature. 346:818-22 (1990); Tuerk and Gold, Science. 249:505-10 (1990)) can be used to find such aptamers. Aptamers have many diagnostic and clinical uses; for almost any use in which an antibody has been used clinically or diagnostically, aptamers too may be used. In addition, aptamers are less expensive to manufacture once they have been identified and can be easily applied in a variety of formats, including administration in pharmaceutical compositions, bioassays and diagnostic tests (Jayasena, Clin Chem. 45:1628-50 (1999)).

In the competitive ELISA binding assay described above, the screen for candidate aptamers includes incorporating the aptamers into the assay and determining their ability to modulate Omi PDZ domain:ligand binding.

4. Antibodies (Abs)

Any antibody that modulates (e.g., inhibits) ligand:Omi PDZ domain binding can be a modulator (e.g., inhibitor) of Omi PDZ domain-ligand interaction. Examples of suitable antibodies include polyclonal, monoclonal, single-chain, anti-idiotypic, chimeric Abs, or humanized versions of such antibodies or fragments thereof. Antibodies may be from any suitable source, including of synthetic origin and any species in which an immune response can be raised.

### Screening Methods

This invention encompasses methods of screening compounds to identify those that modulate Omi PDZ-ligand interaction. Screening assays are designed to identify compounds that bind or complex with Omi PDZ and/or ligand, or otherwise interfere with the interaction of Omi PDZ and cellular factors. One approach to determining the ability of a candidate compound to be a modulator is to assess the activity of the candidate compound in a competitive inhibition assay in the presence of a known Omi PDZ binder, such as any of the binder peptides (e.g., the high affinity binders described in the Examples) disclosed herein. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for modulators are common in that they call for contacting the drug candidate with Omi PDZ (or equivalent thereof) and/or binding ligand that is involved in the binding interaction of Omi PDZ and the binding ligand, under conditions and for a time sufficient to allow these two components to interact.

In binding assays, the interaction is binding and the complex formed can be isolated or detected in the reaction mixture. In a particular embodiment, a candidate substance or molecule is immobilized on a solid phase, e.g., on a microtiter plate, by covalent or non-covalent attachments. Non-covalent attachment generally is accomplished by coating the solid surface with a solution of the substance/molecule and drying. Alternatively, an immobilized affinity molecule, such as an antibody, e.g., a monoclonal antibody, specific for the substance/molecule to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to Omi PDZ or its binding partner, its interaction with the polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

In any of the screening processes above, it is often desirable to assess the modulatory capability of a candidate compound by determining its binding ability to Omi PDZ and a known high affinity binder (such as one of those described herein).

Candidate compounds can be generated by combinatorial libraries and/or mutations of known binders based on information described herein, in particular information relating to contributions and importance to Omi PDZ-ligand binding interactions of individual residues and moieties within a ligand or Omi PDZ sequence itself.

Compounds that interfere with the interaction of Omi PDZ and binding ligand can be tested as follows: usually a reaction mixture is prepared containing Omi PDZ and a ligand under conditions and for a time allowing for the interaction and binding of the two molecules. To test the ability of a candidate compound to inhibit the binding interaction, the reaction is run in the absence and in the presence of the test compound. In addition, a placebo may be added to a third reaction mixture, to serve as positive control. The binding (complex formation) between the test compound and Omi PDZ and/or binding ligand present in the mixture is monitored as described hereinabove. The formation of a complex in the control reaction(s) but not in the reaction mixture containing the test compound indicates that the test compound interferes with the interaction of Omi PDZ and binding ligand.

As described herein, a substance/molecule of the invention can be a peptide. Methods of obtaining such peptides are well known in the art, and include screening peptide libraries for binders to a target antigen. In one embodiment, suitable target antigens would comprise Omi PDZ (or portion thereof that comprises binding site for a Omi PDZ ligand), which is described in detail herein. Libraries of peptides are well known in the art, and can also be prepared according to art methods. See, e.g., Clark et al., U.S. Pat. No. 6,121,416. Libraries of peptides fused to a heterologous protein component, such as a phage coat protein, are well known in the art, e.g., as described in Clark et al., supra. In one embodiment, a peptide having ability to block Omi PDZ protein-protein interaction comprises the amino acid sequence of any of the binder peptides disclosed herein. In another embodiment, a peptide having ability to block Omi PDZ protein-protein interaction comprises the amino acid sequence of a binder peptide obtained from a modulator screening assay as described above. In one embodiment, the peptide has the ability to compete with one or more of the binder peptides disclosed herein (see Examples) for binding to Omi PDZ. In one embodiment, the peptide binds to the same epitope on Omi PDZ to which one or more of the binder peptides disclosed herein (see Examples) bind. Variants of a first peptide binder can be generated by screening mutants of the peptide to obtain the characteristics of interest (e.g., enhancing target binding affinity, enhanced pharmacokinetics, reduced toxicity, improved therapeutic index, etc.). Mutagenesis techniques are well known in the art. Furthermore, scanning mutagenesis techniques (such as those based on alanine scanning) can be especially helpful to assess structural and/or functional importance of individual amino acid residues within a peptide.

Determination of the ability of a candidate substance/molecule of the invention, such as a peptide comprising the amino acid sequence of a binder peptide disclosed herein, to modulate Omi PDZ activity, can be performed by testing the modulatory capability of the substance/molecule in in vitro or in vivo assays, which are well established in the art, e.g., as described in Martins et al. (J. Biol. Chem. 278(49):49417-49427 (2003)) and Faccio et al. (J. Biol. Chem. 275(4):2581-2588 (2000)).

### Examples of Uses for Omi PDZ Binders and Modulators of Omi PDZ-Ligand Interaction

The identification and characterization of the Omi PDZ peptide binders as described herein provide valuable insights into the cellular functions of the Omi protein, and provides compositions and methods for modulating the in vivo interactions between this important cellular protein and its binding partner(s). For example, these peptides and their homologs can be utilized to interfere with the in vivo binding interactions involving Omi PDZ. Homologs can be generated conveniently based on their binding and/or functional characteristics relative to the well-characterized peptides provided herein. These peptides can further be utilized to elucidate cellular and physiological polypeptides that constitute Omi PDZ in vivo complexes. Indeed, as shown by the unexpected results described herein, binding partners of Omi PDZ can be located both in the conventional C-terminal region and also the heretofore unknown N-terminal and/or internal regions of a polypeptide.

As described herein (see, e.g., the Examples), well-characterized high-affinity peptide binders of Omi PDZ can be further used to elucidate important structural characteristics of Omi PDZ itself. Knowledge of such provides for development of modulatory agents based on modification of the Omi PDZ sequence itself. The invention provides Omi PDZ variants as disclosed herein that have enhanced or reduced ability to bind Omi PDZ binding partners. Other variants can be similarly identified.

Omi PDZ-binding partner modulators developed based on the ligand peptides described herein can be used to achieve the modulatory effect of interest. For example, such manipulation may include inhibition of the association between Omi PDZ domain and its cognate binding protein. In another example, such manipulation may include agonistic effects through, for example, induction of cellular functions as a result of binding of the modulators to Omi PDZ or through enhancement of association between Omi PDZ domain and its cognate binding protein by the modulators.

Other uses of modulators of Omi PDZ include diagnostic assays for diseases related to Omi and its associating partners, the use of the Omi PDZ domain and ligands in fusion proteins as purification handles and anchors to substrates.

Identification of binders capable of binding to Omi PDZ domain at varying affinities, as described herein, provide useful avenues for modulating biologically important protein-protein interactions in vivo. As is well-established in the art, the Omi protein is implicated in important biological processes, including regulation of apoptosis and protein quality control in mitochondria. The Omi protein contains a PDZ domain, which is a domain reported to be essential in protein-protein binding interactions. Thus, identification of molecules that are capable of modulating these interactions points to avenues of therapeutic and/or diagnostic applications and strategies that would not be possible in the absence of knowledge of such molecules and interactions. Modulatory compounds (e.g., inhibitory or agonistic) can be delivered into live cells using appropriate routes of administration known in the art, e.g., via microinjection, antenapedia peptide or lipid transfection reagents, to serve as Omi PDZ domain-specific competitive modulators in order to modulate, and in some instances validate the physiological importance of Omi PDZ ligand interaction in a particular tissue, cell, organ or pathological condition. Suitable assays exist to monitor the PDZ ligand interaction and the physiological effect of modulation of said interaction. This does not require that the physiological ligand for Omi PDZ domain is discovered by phage display, only that the modulator is specific for the PDZ domain and of sufficient affinity to disrupt the interaction of said ligand with the PDZ domain. Finally, as with any protein linked with a disease process, one must establish how a drug should affect the protein to achieve therapeutic benefit. Modulatory compounds, such as peptides/ligands, may be delivered into live cells or animal models which are models for a disease (i.e. mimic certain properties of a disease) to determine if disruption of Omi PDZ-ligand interaction by the modulatory compound of interest provides an outcome consistent with expectations for therapeutic benefit.

Methods of detecting protein-protein (or peptide) interactions in vivo are known in the art. For example, the methods described by Michnick et al. in U.S. Pat. Nos. 6,270,964 B1 & 6,294,330 B1 can be used to analyze interactions of Omi PDZ domain-containing protein (including any described herein) and a cognate ligand or synthetic peptide (including any described herein). Furthermore, these methods can be used to assess the ability of a molecule, such as a synthetic peptide, to modulate the binding interaction of Omi PDZ-domain protein and its cognate ligand in vivo.

### Therapeutic/Prophylactic Applications

Compounds that have the property of increasing or decreasing Omi PDZ protein activity are useful. This increase in activity may come about in a variety of ways, for example by administering to a subject in need thereof an effective amount of one or more of the modulators described herein.

“Antagonists” or “negative modulators” include any molecule that partially or fully blocks, inhibits, or neutralizes a biological activity of Omi PDZ and/or its endogenous ligand(s). Similarly, “agonists” or “positive modulators” include any molecule that mimics or enhances a biological activity of Omi PDZ and/or its endogenous ligand(s). Molecules that can act as agonists or antagonists include the modulators of Omi PDZ-binder/ligand interaction described herein, including but not limited to Abs or antibody fragments, fragments or variants of Omi PDZ/ligands/binders, peptides, small organic molecules, etc.

The invention provides various methods based on the discovery of various binding molecules capable of interacting specifically with Omi PDZ, and the identification of unique characteristics of the binding interactions between Omi PDZ and ligand binding peptides.

Various substances or molecules (including peptides, etc.) may be employed as therapeutic agents. These substances or molecules can be formulated according to known methods to prepare pharmaceutically useful compositions, whereby the product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a substance or molecule of the invention is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. No. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a substance or molecule is desired in a formulation with release characteristics suitable for the treatment of any disease or disorder requiring administration of the substance or molecule, microencapsulation of the substance or molecule is contemplated. Microencapsulation of recombinant proteins for sustained release has been successfully performed with human growth hormone (rhGH), interferon- (rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

### Pharmaceutical Compositions

A modulator molecule/substance of the invention can be incorporated into compositions, which in some embodiments are suitable for pharmaceutical use. Such compositions typically comprise the nucleic acid molecule, peptide/protein, small molecule and/or antibody, and an acceptable carrier, for example one that is pharmaceutically acceptable. A “pharmaceutically acceptable carrier” includes any and all solvents, dispersion media, coatings, antibacterial and antifungal agents, isotonic and absorption delaying agents, and the like, compatible with pharmaceutical administration (Gennaro, Remington: The science and practice of pharmacy. Lippincott, Williams & Wilkins, Philadelphia, Pa. (2000)). Examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

1. General Considerations

A pharmaceutical composition is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents; for example, parabens, chlorobutanol, phenol, ascorbic acid, and thimerosal, can contain microorganism contamination. Isotonic agents; for example, sugars, polyalcohols such as mannitol, sorbitol, and sodium chloride can be included in the composition. Compositions that can delay absorption include agents such as aluminum monostearate and gelatin.

Sterile injectable solutions can be prepared by incorporating the active compound (e.g., any modulator substance/molecule of the invention) in the required amount in an appropriate solvent with one or a combination of ingredients as required, followed by sterilization. Generally, dispersions are prepared by incorporating the active compound into a sterile vehicle that contains a basic dispersion medium, and the other required ingredients. Sterile powders for the preparation of sterile injectable solutions, methods of preparation include vacuum drying and freeze-drying that yield a powder containing the active ingredient and any desired ingredient from a sterile solutions.

3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

4. Compositions for Inhalation

For administration by inhalation, the compounds are delivered as an aerosol spray from a nebulizer or a pressurized container that contains a suitable propellant, e.g., a gas such as carbon dioxide.

5. Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

6. Carriers

In one embodiment, the active compounds are prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art. Liposomal suspensions can also be used as pharmaceutically acceptable carriers. These can be prepared according to methods known to those skilled in the art, such as in (Eppstein et al., U.S. Pat. No. 4,522,811, 1985).

7. Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms are dictated by, and directly dependent on, the unique characteristics of the active compound and the particular desired therapeutic effect, and the inherent limitations of compounding the active compound.

8. Gene Therapy Compositions

The nucleic acid molecules can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., Proc Natl Acad Sci USA. 91:3054-7 (1994)). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

9. Dosage

The pharmaceutical composition and method may further comprise other therapeutically active compounds that are usually applied in the treatment of Omi protein-related (specifically Omi PDZ-related) conditions.

In the treatment or prevention of conditions which require Omi PDZ-ligand modulation, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

10. Kits for Compositions

The compositions (e.g., pharmaceutical compositions) can be included in a kit, container, pack, or dispenser together with instructions for administration. When supplied as a kit, the different components of the composition may be packaged in separate containers and admixed immediately before use. Such packaging of the components separately may permit long-term storage without losing the active components' functions.

Kits may also include reagents in separate containers that facilitate the execution of a specific test, such as diagnostic tests or tissue typing.

(a) Containers or Vessels

The reagents included in kits can be supplied in containers of any sort such that the life of the different components are preserved and are not adsorbed or altered by the materials of the container. For example, sealed glass ampules may contain lyophilized modulator substance/molecule and/or buffer that have been packaged under a neutral, non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, laserdisc, audio tape, etc. Detailed instructions may not be physically associated with the kit; instead, a user may be directed to an Internet web site specified by the manufacturer or distributor of the kit, or supplied as electronic mail.

The following examples are included to demonstrate preferred embodiments of the present invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing form the spirit and scope of the invention.

### EXAMPLES

### Experimental Procedures

Materials—Enzymes were from New England Biolabs. Maxisorp immunoplates and 384-well assay plates were from Nalge NUNC International (Naperville, Ill.). E. coli XL1-Blue, Escherichia coli BL21 and KO7 were from Stratagene. Plasmid pET15b was from Novagen. Thrombin was from Calbiochem. Bovine serum albumin (BSA) and Tween 20 were from Sigma. HRP/anti-M13 antibody conjugate, HRP/anti-GST antibody conjugate, glutathione Sepharose-4B, plasmid pGEX6P-3 and Superdex-75 were from Amersham Pharmacia Biotech. NiNTA was from Qiagen. 3,3′,5,5′-Tetramethyl-benzidine/H₂O₂ (TMB) peroxidase substrate was from Kirkegaard and Perry Laboratories Inc. NeutrAvidin was from Pierce Biotechnology Inc.

Oligonucleotide synthesis—Oligonucleotides for combinatorial scanning were designed as described previously using equimolar DNA degeneracies(17). The particular mutagenic oligonucleotides are listed in Table 1.

TABLE I
Table I Mutagenic oligonucleatides for constructing libraries.

| | |
|---|---|
| Oligo to construct libC | |
| ATC GAC AGC GCC CCC GGT GGC GGA NNK NNK NNK NNK NNK NNK | (SEQ ID NO:20) |
| NNK NNK NNK NNK TGA TAA ACC GAT ACA | |
| Oligos to make stop template for Shtogun scanning | |
| CTG GGC AGC CTC GAG TAA TAA TAA CGA GAA CCA AAC TTT | (SEQ ID NO:21) |
| GCT GAA CTA CAG CTT TAA TAA TAA GCA CAC CGG GCT GGT | (SEQ ID NO:22) |
| TTG GCC ATT GGG GAG TAA TAA TAA CAG ATC CGG CGG GGA | (SEQ ID NO:23) |
| Oligos to construct shotgun scanning libraries | |
| CTG GGC AGC CTC GAG SST SST KMT RYT GST GYT RYG RYG SYT RCT | (SEQ ID NO:24) |
| SYT KCC SCA KCC RYT SYT GCT GMA SYT SMA SYT CGA GAA CCA AAC | |
| TTT | |
| GCT GAA CTA CAG CTT SST GMA SCA RMC KYT SCA GMT GYT SMA | (SEQ ID NO:25) |
| SMT GST GYT SYT RYT SMT RMA GYT RYT SYT GST KCC SCA GCA CAC | |
| CGG GCT GGT | |
| TTG GCC ATT GGG GAG SMA RYG GYT SMA RMC GCT GMA GMT GYT | (SEQ ID NO:26) |
| KMT GMA GCT GYT SST RCT SMA KCC SMA SYT GCA GYT CAG ATC | |
| CGG CGG GGA | |

DNA degeneracies are represented by IUB code (K = G/T, M = A/C, N = A/C/G/T, R = A/G, S = G/C, W = A/T, Y = C/T)

Synthetic Peptides—Peptides were synthesized using standard 9-fluorenylmethoxycarbonyl (Fmoc) protocols, cleaved off the resin with 2.5% triisopropylsilane and 2.5% H₂O in trifluoroacetic acid (TFA), and purified by reversed-phase high performance liquid chromatography (HPLC). The purity and mass of each peptide were verified by liquid chromatography/mass spectrometry (LC/MS).

Expression and purification of GST-hOmiPDZ: DNA fragment encoding hOmi PDZ domain was cloned into BamHI/XhoI sites of pGEX6P-3 plasmid, creating a GST-hOmiPDZ fusion protein. Single colony of E. Coli. BL21(DE3) harboring the expressing plasmid was inoculated into 30 ml LB medium supplemented with 50 μg/ml Carbenicilin and was grown at 37° C. for overnight. The bacteria were harvested, washed, resuspended and inoculated into 500 ml LB/carb medium. The culture was grown at 37° C. to mid-log phase (A₆₀₀=0.8). Protein expression was induced with 0.4 mM IPTG and grown at 30° C. for 16 h. The bacteria were pelleted by centrifugation at 4000 g for 15 minutes, washed with PBS for twice and frozen at −80° C. for at least 8 h. The pellet was then resuspended in 50 ml PBS and lyzed by passing through the Microfluidizer® Processing Equipment. The GST-hOmiPDZ was purified from cell lysate with affinity chromatography on 2 ml of glutathione Sepharose-4B according to manufactory manual.

Selection for Omi PDZ peptides—Previously described procedures were used to isolate peptides that bound to a GST-Omi PDZ fusion, using libraries of random decapeptides fused to either the C terminus(18) or octapeptide fused to the N terminus(19) of the M13 gene-8 major coat protein, designated as libC or libN, respectively. After three rounds of selection, individual clones were grown in a 96-well format in 500 μL of 2YT broth supplemented with carbenicillin, kanamycin and KO7, and the culture supernatants were used directly in phage ELISAs(19) to detect peptides that bound specifically to Omi PDZ. Peptide sequences derived from positive clones were aligned and tabulated. A total of 95 positive colonies from libC and 89 from libN were analyzed. (see Table II). Table II shows sequences of phage-displayed selectants. The sequences were selected after three rounds of sorting with IPTG induction. Hydrophobic residues (A, F, I, L, M, V, W, Y) are italicized and bolded, n is the number of siblings.

TABLE II

| Position | | | | | | | | | | |
|---|---|---|---|---|---|---|---|---|---|---|
| −9 | −8 | −7 | −6 | −5 | −4 | −3 | −2 | −1 | 0 | n |

peptides derived from libC

| | | | | | | | | | | | |
|---|---|---|---|---|---|---|---|---|---|---|---|
| K | [symbol] | [symbol] | S | [symbol] | T | [symbol] | [symbol] | [symbol] | [symbol] | 42 | (SEQ ID NO:2) |
| [symbol] | [symbol] | D | R | [symbol] | P | H | [symbol] | [symbol] | [symbol] | 15 | (SEQ ID NO:3) |
| P | G | R | [symbol] | G | P | [symbol] | [symbol] | [symbol] | [symbol] | 2 | (SEQ ID NO:27) |
| D | S | [symbol] | [symbol] | [symbol] | D | [symbol] | [symbol] | [symbol] | [symbol] | 1 | (SEQ ID NO:28) |
| N | Q | R | [symbol] | [symbol] | [symbol] | [symbol] | [symbol] | [symbol] | I | 1 | (SEQ ID NO:29) |
| | S | S | [symbol] | [symbol] | R | [symbol] | [symbol] | [symbol] | [symbol] | 1 | (SEQ ID NO:30) |
| D | R | [symbol] | N | [symbol] | [symbol] | [symbol] | [symbol] | [symbol] | I | 1 | (SEQ ID NO:31) |
| [symbol] | P | T | [symbol] | [symbol] | T | [symbol] | [symbol] | [symbol] | [symbol] | 1 | (SEQ ID NO:32) |
| [symbol] | [symbol] | S | [symbol] | [symbol] | R | [symbol] | [symbol] | [symbol] | [symbol] | 1 | (SEQ ID NO:33) |
| [symbol] | [symbol] | G | [symbol] | [symbol] | E | [symbol] | [symbol] | [symbol] | [symbol] | 1 | (SEQ ID NO:34) |
| S | [symbol] | [symbol] | [symbol] | [symbol] | R | [symbol] | [symbol] | [symbol] | [symbol] | 1 | (SEQ ID NO:35) |
| T | [symbol] | S | D | [symbol] | [symbol] | [symbol] | [symbol] | [symbol] | [symbol] | 1 | (SEQ ID NO:36) |
| [symbol] | [symbol] | G | T | [symbol] | [symbol] | [symbol] | P | H | [symbol] | 1 | (SEQ ID NO:37) |
| T | R | [symbol] | N | [symbol] | [symbol] | [symbol] | [symbol] | [symbol] | [symbol] | 1 | (SEQ ID NO:38) |
| R | [symbol] | P | [symbol] | [symbol] | [symbol] | [symbol] | [symbol] | [symbol] | [symbol] | 1 | (SEQ ID NO:39) |
| S | K | [symbol] | R | [symbol] | [symbol] | [symbol] | [symbol] | [symbol] | [symbol] | 1 | (SEQ ID NO:40) |
| T | G | [symbol] | S | [symbol] | T | [symbol] | [symbol] | F | [symbol] | 1 | (SEQ ID NO:41) |
| S | [symbol] | [symbol] | N | [symbol] | [symbol] | [symbol] | [symbol] | [symbol] | [symbol] | 1 | (SEQ ID NO:42) |
| G | [symbol] | [symbol] | P | [symbol] | [symbol] | [symbol] | [symbol] | [symbol] | [symbol] | 1 | (SEQ ID NO:43) |
| T | [symbol] | H | S | [symbol] | [symbol] | [symbol] | [symbol] | [symbol] | [symbol] | 1 | (SEQ ID NO:44) |
| [symbol] | [symbol] | D | S | [symbol] | P | I | [symbol] | [symbol] | [symbol] | 1 | (SEQ ID NO:45) |
| I | P | L | H | [symbol] | [symbol] | [symbol] | [symbol] | [symbol] | [symbol] | 1 | (SEQ ID NO:46) |
| | | | R | [symbol] | T | [symbol] | [symbol] | [symbol] | [symbol] | 1 | (SEQ ID NO:47) |
| | | | [symbol] | [symbol] | R | [symbol] | [symbol] | [symbol] | [symbol] | 1 | (SEQ ID NO:48) |

peptides derived from libN

| | | | | | | | | | | |
|---|---|---|---|---|---|---|---|---|---|---|
| [symbol] | E | [symbol] | z,57 | G | z,58 | E | [symbol] | G | 31 | (SEQ ID NO:4) |
| S | H | [symbol] | [symbol] | G | G | [symbol] | [symbol] | G | 17 | (SEQ ID NO:49) |
| [symbol] | T | E | [symbol] | [symbol] | [symbol] | G | [symbol] | G | 10 | (SEQ ID NO:50) |
| G | [symbol] | [symbol] | G | [symbol] | [symbol] | [symbol] | D | G | 9 | (SEQ ID NO:7) |
| E | S | [symbol] | [symbol] | [symbol] | G | [symbol] | E | G | 7 | (SEQ ID NO:8) |
| G | G | [symbol] | [symbol] | [symbol] | G | P | A | G | 5 | (SEQ ID NO:9) |
| [symbol] | G | D | S | [symbol] | [symbol] | [symbol] | G | G | 4 | (SEQ ID NO:10) |
| [symbol] | G | [symbol] | [symbol] | [symbol] | G | P | G | G | 1 | (SEQ ID NO:51) |
| S | T | D | [symbol] | [symbol] | [symbol] | G | [symbol] | G | 1 | (SEQ ID NO:52) |
| G | D | [symbol] | [symbol] | [symbol] | T | [symbol] | G | G | 1 | (SEQ ID NO:53) |
| S | S | D | [symbol] | [symbol] | [symbol] | G | [symbol] | G | 1 | (SEQ ID NO:54) |
| G | [symbol] | [symbol] | [symbol] | [symbol] | [symbol] | [symbol] | D | G | 1 | (SEQ ID NO:55) |
| [symbol] | [symbol] | [symbol] | G | [symbol] | [symbol] | [symbol] | D | G | 1 | (SEQ ID NO:56) |

Construction of Libraries for Omi PDZ Shotgun Scanning—Omi PDZ was displayed on the surface of M13 bacteriophage by modifying a previously described phagemid (pS2202b) (20). Standard molecular biology techniques were used to replace the fragment of pS2202b encoding Erbin PDZ with a DNA fragment encoding Omi PDZ. The resulting phagemid (p8hOmi) contained an open reading frame that encoded the maltose binding protein secretion signal, followed by an epitope tag (amino acid sequence: SMADPNRFRGKDLGS (SEQ ID. NO:19)), followed by Omi PDZ and ending with the M13 gene-8 coat protein. E. Coli. harboring p8hOmi were co-infected with KO7 helper phage and grown at 30° C. with 25 mM IPTG induction, resulting in the production of phage particles that encapsulated p8hOmi DNA and displayed Omi PDZ in a multivalent format.

Libraries were constructed using previously described methods(17) with appropriately designed “stop template” versions of p8hOmi. For each library, we used a stop template that contained TAA stop codons within each of the regions to be mutated. The stop template was used as the template for the Kunkel mutagenesis method(21) with mutagenic oligonucleotides (see above) designed to simultaneously repair the stop codons and introduce mutations at the desired sites.

For shotgun scanning, wild-type codons were replaced with corresponding degenerate codons shown in Table 1 of Vajdos et al(22). Three separate libraries were constructed with each library designed to mutate 21, 22 or 18 Omi PDZ residues with no overlap among the three. Libraries L1, L2 and L3 were constructed with mutagenic oligonucleotides SGL1, SGL2 and SGL3, respectively. Library L1 and L2 mutated residues in two continuous stretches of sequence between positions 226-246 and 247-268 while library L3 mutated residues between positions 286-306. The library diversities were as follows: L1, 7.8×10¹⁰; L2 5.5×10¹⁰; L3, 4.0×10¹⁰.

Library Sorting and Analysis—Phage from the libraries described above were propagated in E. coli XL1-blue with the addition of KO7 helper phage. After overnight growth at 37° C. (for peptide library) or 30° C. (for shotgun library), phage were concentrated by precipitation with PEG/NaCl and resuspended in PBS, 0.5% BSA, 0.1% Tween 20, as described previously(19). Phage solutions (10¹² phage/mL) were added to 96-well maxisorp immunoplates that had been coated with capture target and blocked with BSA. For shotgun library sorting, two different targets were used; for the display selection the target was an immobilized antibody that recognized the epitope tag fused to the N terminus of Omi PDZ, while for the function selection a biotinylated peptide that binds to Omi PDZ with high affinity (biotin-SWTMFWV) was immobilized on NeutrAvidin-coated plates. Following a 2 h incubation to allow for phage binding, the plates were washed 10 times with PBS, 0.05% Tween 20. Bound phage were eluted with 0.1 M HCl for 10 min and the eluent was neutralized with 1.0 M Tris base. Eluted phage were amplified in E. Coli XL1-blue and used for further rounds of selection.

Individual clones from each round of selection were grown in a 96-well format in 500 μL of 2YT broth supplemented with carbenicillin, kanamycin and KO7, and the culture supernatants were used directly in phage ELISAs(19) to detect phage-displayed Omi PDZ variants that bound to either biotin-SWTMFWV or anti-tag antibody. After two rounds of selection, clones from L1, L2 and L3 that exhibited positive phage ELISA signals at least two-fold greater than signals on control plates coated with BSA were considered as positive clones and they were subjected to DNA sequence analysis (see below).

The sequences were analyzed with the program SGCOUNT as described previously(17). SGCOUNT aligned each DNA sequence against the wild-type DNA sequence by using a Needleman-Wunch pairwise alignment algorithm, translated each aligned sequence of acceptable quality, and tabulated the occurrence of each natural amino acid at each position. For the function selection, the number of analyzed clones are indicated in parenthesis following the name of each library: L1 (93), L2 (100), L3 (88). For the display selection, the following number of clones were analyzed: L1 (102), L2 (77), L3 (93).

DNA Sequencing—Culture supernatants containing phage particles were used as templates for PCRs that amplified DNA fragments containing the Omi PDZ gene, and these fragments were sequenced as described previously(22).

Affinity Assays—The binding affinities of peptides for Omi PDZ were determined as IC₅₀ values using a previously described competition ELISA(18). The IC₅₀ value was defined as the concentration of peptide that blocked 50% of PDZ domain binding to immobilized peptide. Assay plates were prepared by immobilizing an amino-terminally biotinylated peptide (biotin-GWTMFWV) on maxisorp plates coated with NeuTravidin and blocked with BSA. A fixed concentration of GST-Omi PDZ fusion protein (20 nM) in PBS, 0.5% BSA, 0.1% Tween 20 (PBT buffer) was preincubated for 1 h with serial dilutions of peptide and then transferred to the assay plates. After 1 h incubation, the plates were washed with PBS, 0.05% Tween 20, incubated for 30 min with HRP/anti-GST antibody (1:10,000) in PBT buffer, washed again, and detected with 3,3′,5,5′-Tetramethyl-benzidine/H₂O₂ (TMB) peroxidase substrate.

Molecular modeling—The high affinity ligand SWTMFWV was built and rendered as β sheet with Biopolymer (Accelrys, Inc.; San Diego, Calif., USA) and optimized with molecular mechanics calculations performed by Discover (Accelrys, Inc.; San Diego, Calif., USA). The ligand was docked to Omi PDZ based on the published coordinates of HtrA2/Omi and Erbin PDZ-ligand complex (Protein Data Bank entry code 1LCY and 1N7T) using Docking module, and the binding was evaluated by Van der Waals and coulomb interaction. The modeled Omi PDZ-ligand complex was finally energy-minimized with Discover using cff91 forcefield. All the above-mentioned modules were implemented under InsightII (Accelrys, Inc.; San Diego, Calif., USA) environment.

### Results

Selection for Omi PDZ Ligands—A library (libC) of random peptides fused to the carboxy terminus of P8 was constructed as described previously(18). The library contained ten degenerate codons (NNK), which predominantly encoded decapeptides, but the possible occurrence of amber stop codons also provided for the display of shorter peptides. The library contained approximately 2.5×10¹⁰ unique members.

Omi PDZ was purified as glutathione S-transferase (GST) fusions from E. coli, and the phage-displayed peptide library was sorted for three rounds of selection against this domain. Transcription of the phagemid encoded P8 gene is regulated by the lac repressor, and display could thus be increased by the addition of 25 mM IPTG.

95 clones after three rounds of selection were sequenced. The variety of sequences ranges in length from seven to ten residues (Table II). The carboxy-terminal residues showed no strong consensus in primary sequence, but demonstrated characteristics of two hydrophobic moieties separated with 1-2 amino acids. While some of the sequences were represented by unique clones, two sequences appeared dominantly with 42 (KVAS WTMFWV (SEQ ID. NO:2)) and 15 siblings (WLDRFPHFWV (SEQ ID. NO:3)).

A library (libN) of random octapeptides fused to N-terminus of P8 was also constructed as described previously(19), and was sorted against Omi PDZ for three rounds of selection. 89 clones were sequenced and 14 unique sequences were obtained. Four of these sequences appeared with multiple clones, which were considered as high affinity ligands for Omi PDZ. The peptide sequences derived from libN showed no similarity to those derived from libC. But they also present the characteristics of two hydrophobic moieties separated with 1-3 residues or single stretch of 3-5 hydrophobic residues (Table II).

Specificity of Peptide Binding—Peptides corresponding to the selected sequences represented by multiple clones were synthesized and assayed for binding affinities. One peptide derived from libC (SWTMFWV (SEQ ID. NO:11)) bound to Omi PDZ with high affinity at IC₅₀=3.3 μM, while the other peptide (RFPHFWV (SEQ ID. NO:12)), which contains more hydrophilic residues, bound to Omi PDZ with 50-fold lower affinity. Amidation of carboxyl terminus of peptide SWTMFWV (SEQ ID. NO:11) abolished its binding to the domain completely, demonstrating the importance of interaction between Omi PDZ and the terminal carboxylate of this ligand. To investigate the energetic contribution of different residues at each position, the relative affinities of serial peptides with Ala replacing the individual residues of peptide SWTMFWV (SEQ ID. NO:11) were measured (Table III). Alanine replacement at Val⁰ and Met⁻³ had a slight impact on binding affinities, with IC₅₀ values increased 3 and 8 fold, respectively. Replacement at Phe⁻² caused moderate loss of affinity by 15 fold. Replacement at Thr⁻⁴ and Ser⁻⁶ had no impact on ligand binding at all. Trptophan is the most preferable residue at position −1 as indicated by the phage selection data (Table II). Replacing it with alanine almost abolished the peptide binding completely. At position −5, tryptophan as well as several other hydrophobic residues with bulky side chains (e.g. Leu and Phe) were preferably selected by phage selection (Table II). Consistently, the replacement of Trp⁻⁵ with alanine caused dramatic loss of binding (>30 fold). To determine the minimal peptide that is required for Omi PDZ binding, the affinities of a series of truncated forms of SWTMFWV (SEQ ID. NO:11) were measured (Table III). Deletion of the first residue, Ser⁻⁶ had no impact on ligand binding. Truncation of the second residue, Trp⁻⁵, resulted in a pentapeptide with 15 fold lower affinity. Further truncation of Thr⁻⁴ increased the binding affinity slightly, whereas truncation up to Met⁻³ resulted in a tripeptide (FWV) with over 30 fold lower binding affinity, which was similar to the peptide with Ala replacement at Trp⁻⁵ (Table III).

Peptides derived from libN with carboxyl terminus blocked by amidation bound to Omi PDZ with affinities in the 15-70 μM range (Table III), indicating that Omi PDZ is able to bind certain peptides with reasonable affinity without terminal carboxylate involved in interaction. To pinpoint the minimal length that is required for ligand-Omi PDZ binding, a series of truncated peptides based on SHWWGGWLG (SEQ ID. NO:5), which shows the highest affinity among ligands derived from LibN, were synthesized and the relative affinities were measured (Table III). Deletion of Ser-8 reduced the binding affinity slightly by three fold, whereas removal of His⁻⁷ or up to Trp⁻⁶ abolished the ligand binding capability completely, indicating that the minimal sequence from the N terminal side should start with His⁻⁷. From the C terminal side, the deletion of Gly⁰ and Leu⁻¹ did not have a detectable effect on binding affinity, and deletion of Trp⁻² abolished the binding of the ligand completely, indicating the importance of the contribution of this residue to ligand-PDZ interaction. As expected, the ligand with deletion up to Gly⁻³ showed no binding to Omi PDZ. Further deletion of Gly⁻⁴, which resulted in a tetrapeptide (SHWW (SEQ ID. NO:18)), resumed the ligand binding to the PDZ domain, although with 3-fold lower affinity. The tripeptide (HWW) appeared to be the minimal sequence for Omi PDZ binding with comparable affinity as tetrapeptide SHWW (SEQ ID. NO:18).

TABLE III

Position

| | | | | | | | | | | |
|---|---|---|---|---|---|---|---|---|---|---|
| Peptide ID | −8 | −7 | −6 | −5 | −4 | −3 | −2 | −1 | 0 | IC50 (μM) |

peptides from libC

| | | | | | | | | | |
|---|---|---|---|---|---|---|---|---|---|
| hOmi_c2 | R | F | P | H | F | W | V | (SEQ ID NO:12) | 161.7 ± 37.2 |
| hOmi_c1 | S | W | T | M | F | W | V | (SEQ ID NO:11) | 3.3 ± 0.4 |
| hOmi_c3 | S | W | T | M | F | W | V-CONH₂ | (SEQ ID NO:57) | NDI |
| hOmi_c1_A1 | S | W | T | M | F | W | A | (SEQ ID NO:58) | 10.6 ± 3.5 |
| hOmi_c1_A2 | S | W | T | M | F | A | V | (SEQ ID NO:59) | >1 mM |
| hOmi_c1_A3 | S | W | T | M | A | W | V | (SEQ ID NO:60) | 48.7 ± 10.9 |
| hOmi_c1_A4 | S | W | T | A | F | W | V | (SEQ ID NO:61) | 24.6 ± 4.1 |
| hOmi_c1_A5 | S | W | A | M | F | W | V | (SEQ ID NO:62) | 3.0 ± 0.9 |
| hOmi_c1_A6 | S | A | T | M | F | W | V | (SEQ ID NO:63) | 107.3 ± 40.1 |
| hOmi_c1_A7 | A | W | T | M | F | W | V | (SEQ ID NO:64) | |
| hOmi_c1_t1 | | W | T | M | F | W | V | (SEQ ID NO:17) | |
| hOmi_c1_t2 | | | T | M | F | W | V | (SEQ ID NO:65) | 46.5 ± 16.7 |
| hOmi_c1_t3 | | | | M | F | W | V | (SEQ ID NO:66) | 24.1 ± 4.1 |
| hOmi_c1_t4 | | | | | F | W | V | | 118.0 ± 32.1 |

peptides from libN

| | | | | | | | | | | | |
|---|---|---|---|---|---|---|---|---|---|---|---|
| hOmi_n4 | A | T | E | F | W | W | G | V | G | (SEQ ID NO:6) | 32.4 ± 4.3 |
| hOmi_n1 | S | H | W | W | G | G | W | L | G | (SEQ ID NO:5) | 21.6 ± 4.0 |
| hOmi_n1_t1 | S | H | W | W | G | G | W | L | | (SEQ ID NO:67) | 17.6 ± 3.9 |
| hOmi_n1_t2 | S | H | W | W | G | G | W | | | (SEQ ID NO:13) | 26.0 ± 5.7 |
| hOmi_n1_t3 | S | H | W | W | G | G | | | | (SEQ ID NO:68) | NDI |
| hOmi_n1_t4 | S | H | W | W | G | | | | | (SEQ ID NO:69) | NDI |
| hOmi_n1_t5 | S | H | W | W | | | | | | (SEQ ID NO:18) | 72.2 ± 12.2 |
| hOmi_n1_t6 | | H | W | W | G | G | W | L | G | (SEQ ID NO:70) | 60.5 ± 20.1 |
| hOmi_n1_t7 | | | W | W | G | G | W | L | G | (SEQ ID NO:71) | NDI |
| hOmi_n1_t8 | | | | W | G | G | W | L | G | (SEQ ID NO:72) | NDI |
| hOmi_n1_t9 | | H | W | W | | | | | | | 107.5 ± 32.2 |
| hOmi_n1_A1 | S | H | W | W | G | G | A | L | | (SEQ ID NO:73) | 175.6 ± 37.2 |
| hOmi_n1_A2 | S | H | W | A | G | G | W | L | | (SEQ ID NO:74) | NDI |
| hOmi_n1_A3 | S | H | A | W | G | G | W | L | | (SEQ ID NO:75) | NDI |
| hOmi_n1_A4 | S | A | W | W | G | G | W | L | | (SEQ ID NO:76) | 75.5 ± 17.2 |

peptides ligand reported previodsly

| | | | | | | | | | | |
|---|---|---|---|---|---|---|---|---|---|---|
| Mxi2 | M | D | I | E | L | V | M | I | (SEQ ID NO:16) | >500 |
| PDZ-Opt | | | G | Q | Y | Y | F | V | (SEO ID NO:14) | 17.6 ± 5.6 |

the ligand with deletion up to Gly⁻³ showed no binding to Omi PDZ. Further deletion of Gly⁻⁴ which resulted in a tetrapeptide (SHWW), resumed the ligand binding to the PDZ domain, although with 3-fold lower affinity. The tripeptide (HWW) appeared to be the minimal sequence for Omi PDZ binding with comparable affinity as tetrapeptide SHWW.

C-terminal sequence of Mxi2 up to 7 residues has been reported to interact with Omi PDZ by immunoprecipitation and yeast two-hybrid assay(1). The binding affinity of this ligand to Omi PDZ was measured with ELISA competition assay. The binding affinity turned out to be very low (IC₅₀>500 μM), which is biologically insignificant. PDZ-opt is an optimized peptide ligand of Omi PDZ derived from a chemically synthesized peptide library(11) with totally different sequence motif from that of the ligands derived from phage libraries. The binding affinity of this ligand is around 20 μM, which is comparable to those derived from phage library libN, but 5-fold lower than the optimized peptide derived from libC (Table III). Table III shows IC₅₀ values for Omi PDZ-binding synthetic peptides. The IC₅₀ values are the mean concentrations of peptide that blocked 50% of Omi PDZ binding to an immobilized high affinity peptide ligand in an ELISA. Peptides from libC and peptides Mxi2 and PDZ-opt were synthesized with acetylated N termini and free C termini, unless indicated otherwise. Peptides from libN were synthesized with free N termini and amidation at C termini.

Molecular modeling of Omi PDZ-ligand interaction—The high affinity ligand SWTMFWV (SEQ ID NO:11) was docked to Omi PDZ based on the published coordinates of HtrA2/Omi(16) and Erbin PDZ-ligand complex(20). In the modeled structure, the peptide ligand forms a β sheet that intercalates between β2 and α3 of the PDZ domain, extending the antiparallel β sheet formed by β2 and β3 of the protein. The terminal carboxylate of the peptide locates in proximate to Tyr228, Ile229, Gly230 and Val231, which correspond to the highly conserved carboxylate binding loop in other PDZ domains(23,24). Val⁰ of the ligand resides close to a well-defined hydrophobic pocket composed by Tyr228, Ile229, Gly230 and Val231. The backbone amide proton of Val231 is directed toward carboxylate oxygen atoms of Val⁰ and can form a hydrogen bond. Bulky side chains of Trp⁻¹ and Phe⁻² present steric hindrance with side chains of Met232, Met233 and Tyr295 on protein. This steric hindrance results in two effects on Omi PDZ-ligand interaction: first, it renders larger the distance between the terminal carboxylate on the peptide and the carboxylate binding loop on the protein compared to that between Erbin PDZ and its ligand; secondly, it brings the residue at the other end of the peptide (position −5) closer to the protein. Met⁻³ and Thr⁻⁴ have no direct interactions with the protein. The side chain of Trp⁻⁵ locates within the Van der Waals interaction distance with another hydrophobic patch on the protein composed by Thr235, Leu236, Ile240 and Leu241. Ser⁻⁶ is solvent-exposed and does not interact with the protein. Thus, this model highlighted that two hydrophobic moieties on ligand, Val⁰Trp⁻¹Phe⁻² and Trp⁻⁵, contribute importantly to Omi PDZ-ligand interaction. This is quite different from any known patterns of PDZ domain-ligand interaction reported previously, in which terminal carboxylates are crucial for the binding and residues at position −5 and up contribute little for ligand-PDZ domain binding. The peptide binding site identified by this model is adjacent to the area where Omi PDZ domain packed against its protease domain(16). This is consistent with the notion that the protease activity of Omi is regulated by its PDZ domain via PDZ ligand binding(11).

Shotgun alanine-scanning of Omi PDZ domain—By superimposing the structure of Omi PDZ with the structure of Erbin PDZ-ligand complex(20), we identified a region that is directly involved or adjacent to the peptide binding site. Three libraries (L1, L2 and L3) were constructed in which 61 residues in and around the peptide binding site (FIG. 1) were represented by trinucleotides that encoded either the wild-type Omi amino acid or alanine (note that due to the particular codons used, some non-alanine mutants were also possible, see Ref(17)). These libraries were then selected for binding to immobilized peptide (biotin-SWTMFWV (SEQ ID. NO:11). Approximately 15%, 40% and 60% of clones from L1, L2 and L3, respectively, showed positive for binding in phage ELISA assays and ˜100 positive clones from each library were sequenced after two rounds of selection. The number of clones with the wild-type residue at each position was compared to the number with each designed mutant (alanine or non-alanine mutants due to the particular codon used) and categorized as substitutions that reduce (ratio >1), do not affect (ratio ˜1), or improve (ratio <1) binding to peptide. To control for variation in expression or display level for different library members, the libraries were also selected for binding to an immobilized antibody capable of recognizing an epitope tag that was displayed at the N-terminus of all library members. The ratio of wild type to mutant in the peptide selection was then scaled by the ratio of wild-type to mutant observed in the antibody selection to give a normalized frequency of occurrence (F; see Table IV). Table IV shows results of Omi PDZ shotgun scan. The wt/mutant ratios were determined from the sequences of binding clones isolated after selection for binding to either a high affinity peptide ligand (function selection) or an anti-tag antibody (display selection). A normalized frequency of occurrence (F) was derived by dividing the function selection wt/mutant ratio by the display selection wt/mutant ratio. In cases where a particular mutation was not observed amongst the function selection sequences, only a lower limit could be defined for the wt/mutant ratio and the F value (indicated by a greater than sign). The F values were determined for alanine substitutions and also for two additional substitutions (m2 and m3) in cases where the alanine scan required a tetranomial codon. The identities of non-alanine substitutions are shown in parentheses to the right of each F value. Bold and italic numbers indicate mutations having more than 16-fold effect on selection; bold only indicates more than 4-fold and less than 16-fold; italic only indicates less than 0.3-fold effect on selection

Most of the residues that are energetically important for ligand binding (F>4) were mutated by L1 library, which explains why only less than 15% colonies from L1 are positive for binding after two rounds of selection.

TABLE IV

| | wt/mutant ratios | | | | | | | | | | |
|---|---|---|---|---|---|---|---|---|---|---|---|
| | function selection | | | display selection | | | F | | | | |
| residue | wt/A | wt/m1 | wt/m2 | wt/A | wt/m1 | wt/m2 | Ala | m1 | | m2 | |
| R226 | 6.5 | 0.7 | 1.1 | 1.1 | 0.7 | 1.0 | 6 | 1 | (G) | 1 | (P) |
| R227 | 5.0 | 3.6 | 0.5 | 1.7 | 1.0 | 0.7 | 3 | 3 | (G) | 0.7 | (P) |
| Y228 | 6.0 | 48 | 1.3 | 0.9 | 0.9 | 0.6 | 7 | 55 | (D) | 2 | (S) |
| I229 | >79 | 79 | 7.2 | 2.3 | 3.5 | 1.2 | >34 | 6 | (T) | 21 | (V) |
| G230 | >91 | | | 3.2 | | | >28 | | | | |
| V231 | 1.3 | | | 1.6 | | | 1 | | | | |
| M232 | 0.7 | 11 | 2.8 | 2.2 | 1.7 | 1.1 | 0.3 | 7 | (T) | 3 | (V) |
| M233 | 0.9 | 5.5 | 0.2 | 4.3 | 2.5 | 2.0 | 0.2 | 2 | (T) | 0.1 | (V) |
| L234 | 4.6 | 9.3 | 0.8 | 1.8 | 1.3 | 1.1 | 3 | 7 | (P) | 0.8 | (V) |
| T235 | 2.8 | | | 0.7 | | | 4 | | | | |
| L236 | 41 | 41 | 8.2 | 2.5 | 1.1 | 1.2 | 16 | 36 | (P) | 7 | (V) |
| S237 | 9.2 | | | 2.6 | | | 4 | | | | |
| P238 | 2.1 | | | 2.1 | | | 1 | | | | |
| S239 | 1.8 | | | 2.4 | | | 1 | | | | |
| I240 | 6.7 | 9.4 | 1.3 | 1.1 | 2.4 | 0.9 | 6 | 4 | (T) | 2 | (V) |
| L241 | 5.4 | 7.2 | 1.1 | 1.3 | 1.2 | 1.1 | 4 | 6 | (P) | 1 | (V) |
| A242 | 1.0 | | | 1.0 | | | 1 | | | | |
| E243 | 0.5 | | | 1.3 | | | 0.4 | | | | |
| L244 | 3.9 | 9.7 | 3.9 | 1.5 | 1.1 | 2.3 | 3 | 9 | (P) | 2 | (V) |
| Q245 | 1.6 | 9.8 | 8.2 | 0.8 | 1.2 | 0.6 | 2 | 5 | (E) | 34 | (P) |
| L246 | 0.8 | 5.6 | 1.2 | 0.9 | 1.3 | 1.0 | 1 | 4 | (P) | 1 | (V) |
| R247 | 2.0 | 1.7 | 6.4 | 0.5 | 0.6 | 0.6 | 4 | 3 | (G) | 10 | (P) |
| E248 | 1.4 | | | 2.2 | | | 1 | | | | |
| P249 | 2.3 | | | 2.3 | | | 1 | | | | |
| S250 | 0.9 | 0.9 | 1.6 | 0.6 | 0.6 | 0.9 | 1 | | | | |
| F251 | 4.7 | 3.5 | 3.5 | 4.2 | 2.0 | 3.5 | 1 | 2 | (S) | 1 | (V) |
| P252 | 1.3 | | | 1.1 | | | 1 | | | | |
| D253 | 1.6 | | | 0.9 | | | 2 | | | | |
| V254 | 4.3 | | | 2.0 | | | 2 | | | | |
| Q255 | 0.8 | 1.8 | 1.5 | 1.2 | 1.8 | 1.1 | 1 | 1 | (E) | 1 | (P) |
| H256 | 2.2 | 30 | 4.9 | 1.1 | 2.3 | 0.8 | 2 | 12 | (D) | 6 | (P) |
| G257 | 3.2 | | | 1.0 | | | 3 | | | | |
| V258 | 12 | | | 4.1 | | | 3 | | | | |
| L259 | >69 | >69 | 2.2 | 14 | 14 | 1.5 | >5 | >5 | (P) | 2 | (V) |
| I260 | 17 | >50 | 1.1 | 15 | 9.7 | 0.7 | 1 | >5 | (T) | 2 | (V) |
| H261 | 0.1 | 5.5 | >11 | 0.4 | 0.8 | 16 | 0.3 | 7 | (D) | >0.7 | (P) |
| K262 | 23 | 47 | >93 | 0.9 | 1.4 | 2.1 | 26 | 34 | (E) | >44 | (T) |
| V263 | 99 | | | 76 | | | 1 | | | | |
| I264 | 0.2 | 2.6 | 1.1 | 1.8 | 1.8 | 1.0 | 0.1 | 2 | (T) | 1 | (V) |
| L265 | 0.9 | 0.5 | 0.8 | 1.8 | 1.4 | 1.3 | 1 | 0.3 | (P) | 0.7 | (V) |
| G266 | 2.7 | | | 2.8 | | | 1 | | | | |
| S267 | 3.7 | | | 3.3 | | | 1 | | | | |
| P268 | 60 | | | 6.5 | | | 9 | | | | |
| Q286 | 1.6 | 0.9 | >32 | 2.0 | 0.8 | 34 | 1 | 1 | (E) | >0.9 | (P) |
| M287 | 2.1 | 1.4 | 0.9 | 2.7 | 2.2 | 1.2 | 1 | 0.7 | (P) | 0.8 | (V) |
| V288 | 7.0 | | | 6.8 | | | 1 | | | | |
| Q289 | 1.1 | 1.9 | 1.7 | 1.0 | 1.4 | 2.7 | 1 | 1 | (E) | 0.6 | (P) |
| N290 | 1.9 | 1.4 | 1.0 | 1.8 | 1.0 | 0.7 | 1 | 1 | (D) | 1 | (T) |
| E292 | 0.7 | | | 0.9 | | | 1 | | | | |
| D293 | 1.1 | | | 1.3 | | | 1 | | | | |
| V294 | 43 | | | 12 | | | 4 | | | | |
| Y295 | 0.2 | >9 | 0.3 | 0.9 | 1.1 | 0.4 | 0.2 | >8 | (D) | 0.8 | (S) |
| E296 | 1.0 | | | 1.0 | | | 1 | | | | |
| V298 | 87 | | | 4.8 | | | 18 | | | | |
| R299 | 0.9 | 3.6 | >36 | 0.7 | 0.7 | 7.7 | 1 | 5 | (G) | >5 | (P) |
| T300 | 1.0 | | | 1.0 | | | 1 | | | | |
| Q301 | 1.3 | 1.6 | 4.9 | 0.9 | 1.1 | 4.7 | 1 | 2 | (E) | 1 | (P) |
| S302 | 2.7 | | | 2.4 | | | 1 | | | | |
| Q303 | 2.4 | 0.7 | 1.6 | 1.3 | 0.5 | 0.7 | 2 | 1 | (E) | 2 | (P) |

The effect of alanine substitutions on peptide binding are indicated in the sequence alignment of human HtrA family (FIG. 1). Several residues that showed significant effect on peptide binding (F>4) upon alanine substitution (Table IV), e.g. Tyr228, Ile229, Gly230 and Thr235, Leu236, S237, Ile240 and Leu241 are located in two patches of residues that are positioned to make favorable contacts with the two hydrophobic epitopes on the ligand (FIG. 1B and FIG. 1C). This is consistent with the model that the ligand interacts with Omi-PDZ via two hydrophobic moieties. Nonetheless, alanine is preferred to the wild type at Met232, Met233 and Tyr295 (F<0.3), indicating the existence of steric hindrance between bulky side chains of Trp⁻¹ and Phe⁻² on ligand and residues Met232, Met233 and Tyr295 on the protein. Alanine substitutions of Val294, Val298 and L304 caused significant detrimental effect on peptide binding. Although these residues are not in or proximate to the peptide binding site, they play important roles on maintaining the α3 conformation that is necessary for the tight ligand binding. Alanine substitutions of some residues in β3, e.g. Leu259, His261, Lys262 and Ile264 were either detrimental (Lys262 and Leu259) or beneficial (His261 and Ile264) to binding, suggesting that they are important for maintaining the β3 conformation that is required for ligand binding.

### Discussion

The peptide ligands described herein were derived from two completely different phage libraries, which is either with a decapeptide fused to the C-terminus of M13 P8 coat protein or an octapeptide fused to the N-terminus of P8. Different from previous findings that most PDZ ligands contain type I or type II consensus binding motifs(25), these peptides do not show strong consensus in sequence. In fact, peptides derived from libC have different types of sequences from those derived from libN, and the sequence of the peptide derived from a chemically synthesized peptide library(11) is also completely different from those described herein. Binder peptides disclosed herein show a common characteristic in sequence: they are highly hydrophobic peptides with at least 6 residues. They contain either two hydrophobic moieties separated by 1-2 residues or a continuous stretch of hydrophobic amino acids. In the former case, one moiety is composed of 2-4 hydrophobic cluster with aromatic residues preferred in at least two positions; the other moiety is composed of one hydrophobic amino acid with bulky side chain, such as Trp, Phe, Leu or Ile (Table II). Such characteristic of Omi PDZ ligands is novel with respect to known PDZ ligand patterns. In particular, peptides without a free C-terminus can also bind to Omi PDZ with reasonably tight affinity, which is also novel with respect to any known ligand-PDZ interaction pattern. These findings represent a new molecular basis for peptide recognition by a PDZ domain.

The peptide specificity study described in Table III sheds light on how Omi PDZ recognizes its ligands. For the peptide series from libC, hOmi_c2 binds to Omi PDZ with 50-fold lower affinity compared to hOmi_c1, and the major sequence difference is the lack of the second hydrophobic moiety in hOmi_c2. The results of alanine scan of hOmi_c1 identified Trp⁻¹ as the most energetically important residue for ligand-PDZ interaction, while Phe⁻² and Met⁻³ contribute moderately to the binding. Separated by Thr⁻⁴, which has no contribution to peptide binding, Trp⁻⁵ is also very energetically important for the binding. Although the replacement of this residue does not abolish ligand binding, it does reduce the affinity dramatically. These results clearly indicate that there are two moieties on hOmi_c1 that contribute significantly to ligand-Omi PDZ interaction. The first moiety is composed of Phe⁻²Trp⁻¹Val⁰ and is required for initial binding, Met⁻³ can enhance such binding moderately by 2-3 folds; the second moiety is composed of Trp⁻⁵ and can enhance the binding affinity dramatically by over 30-fold. The truncation study on hOmi_c1 confirmed the existence of two binding moieties on ligands. Deletion of Trp⁻⁵ caused significant loss of affinity, and the tripeptide FWV showed similar binding affinity as hOmi_c1_A6, in which Trp⁻⁵ was replaced by alanine. The truncation study on the optimized peptide from libN (hOmi_n1) also suggested two binding moieties on the ligand that were composed of His⁻⁷Trp⁻⁶Trp⁻⁵ and Trp⁻² (Table III), corresponding to Phe⁻²Trp⁻¹Val⁰ and Trp⁻⁵ on hOmi_c1, respectively. His⁻⁷ is critical for the peptide binding as shown in Table III. It probably plays a similar role as the C-terminal carboxylate in hOmi_c1, possibly forming a hydrogen bond with a backbone proton on Omi PDZ or making favorable Coulombic interaction.

Molecular modeling of ligand-Omi PDZ complex reveals two hydrophobic patches on the protein locate in such positions that they may interact with the two hydrophobic moieties on the ligands. The shotgun scanning of Omi PDZ confirmed such model (data not shown). In the model, the backbone amide proton of Val231 forms a hydrogen bond with carboxylate oxygen atoms of Val⁰. Although mutating Val231 to Ala did not have a detrimental effect on ligand binding, alanine substitutions of Ile229 and Gly230 do have a dramatic effect (Table IV), suggesting that maintaining the local backbone conformation at this region is critical for peptide binding. In fact, most of the residues identified as energetically significant components for ligand binding (F>3.5) are more important for maintaining the proper conformation that is necessary for tight ligand binding rather than for interacting with the ligand directly. For example, Leu236 (F=16), whose side chain does not direct toward the ligand and is mostly buried under the surface, plays an important role on holding the proper conformation of the hydrophobic patch composed of Thr235 (F=3.8), S237 (F=3.6), Ile240 (F=6) and Leu241 (F=4.3), which interacts with the second hydrophobic moiety on the ligand. Lys262 (F=26) and Leu259 (F=4.8), which are not at vicinity to the peptide binding site, are important to maintain the conformation of the β3 sheet so that the antiparallel interaction between β3/β2 and the ligand could be maintained. In the case of Val298 (F=18) and Leu304 (F=18), they are completely buried under the surface, but the interaction between these two residues are important to maintain the conformation of α2 helix, which is necessary for tight ligand binding.

Alanine scanning of peptide homi_c1 indicated that Trp⁻¹ contributes significantly to the binding. Molecular modelling indicated that side chains of M232 and M233 might interact with Trp⁻¹ in favor of ligand binding. However, shotgun scan data demonstrated that instead of conferring any specific energetic contribution to binding, Met232 (F=0.3) and Met233 (F=0.2) are actually detrimental for the binding. Therefore, the benefit to binding conferred by Trp⁻¹ derives from the Omi PDZ side chains-independent interactions with the backbone of β2 and β3 sheets. This is reminiscent of the ligand-Erbin PDZ interaction, in which the role of tryptophan is to stabilize antiparallel interaction between two β strands(20).

Omi/HtrA2 is highly homologous with bacterial HtrA family, whose protease activity plays a role of disposal of unfolded protein upon heat shock stimulus(2). The serine protease activity of Omi is regulated by its PDZ domain via PDZ-ligand interaction(11,16). The molecular basis for ligand recognition by Omi PDZ described herein is different from most previously reported ligand-PDZ interactions in which 3-5 conserved motif at the C terminus of its binding partner is required(23-30) (one report does suggest that PDZ7 domain of Glutamate receptor interacting protein 1 (GRIP1) could interact with its partner via a hydrophobic patch interaction(31)). The results described herein demonstrate that Omi PDZ can bind to a variety of peptides with stretches of hydrophobic residues, either with free C-terminal or internal sequences, which is also the characteristic of denatured or damaged proteins inside the cell. These results suggest that the in vivo ligands for Omi PDZ likely include unfolded proteins in the intermembrane space of mitochondria. Upon PDZ ligand engagement, the serine protease activity is activated and subsequently degrades the damaged proteins. From an evolutionary point of view, it is reasonable to postulate that a primary function of Omi/HtrA2 is maintaining protein quality in mitochondria, since its bacterial ancestors play a similar role. As described herein, there is also evidence indicating that Omi/HtrA2 can promote apoptosis(4-9,11,14,15). Indeed, it has been speculated that the capability of Omi to promote cell death can be a bonus or secondary function in addition to its primary function in mitochondria protein quality control(32). In the case that the stability of mitochondria could not be maintained under extreme stress, Omi/HtrA2, possibly together with other apoptosis-promoting proteins, such as cytochrome c, Smac/DIABLO, AIF or endonuclease G, could be released into the cytosol and orchestrate to induce apoptosis through either caspase-dependent or caspase-independent pathways.

### PARTIAL LIST OF REFERENCES

1. Faccio, L., Fusco, C., Chen, A., Martinotti, S., Bonventre, J. V., and Zervos, A. S. (2000) J Biol Chem 275, 2581-2588
2. Spiess, C., Beil, A., and Ehrmann, M. (1999) Cell 97, 339-347
3. Gray, C. W., Ward, R. V., Karran, E., Turconi, S., Rowles, A., Viglienghi, D., Southan, C., Barton, A., Fantom, K. G., West, A., Savopoulos, J., Hassan, N. J., Clinkenbeard, H., Hanning, C., Amegadzie, B., Davis, J. B., Dingwall, C., Livi, G. P., and Creasy, C. L. (2000) Eur J Biochem 267, 5699-5710
4. Suzuki, Y., Imai, Y., Nakayama, H., Takahashi, K., Takio, K., and Takahashi, R. (2001) Mol Cell 8, 613-621
5. Martins, L. M., Iaccarino, I., Tenev, T., Gschmeissner, S., Totty, N. F., Lemoine, N. R., Savopoulos, J., Gray, C. W., Creasy, C. L., Dingwall, C., and Downward, J. (2002) J Biol Chem 277, 439-444
6. Martins, L. M. (2002) Cell Death Differ 9, 699-701
7. van Loo, G., van Gurp, M., Depuydt, B., Srinivasula, S. M., Rodriguez, I., Alnemri, E. S., Gevaert, K., Vandekerckhove, J., Declercq, W., and Vandenabeele, P. (2002) Cell Death Differ 9, 20-26
8. Hegde, R., Srinivasula, S. M., Zhang, Z., Wassell, R., Mukattash, R., Cilenti, L., DuBois, G., Lazebnik, Y., Zervos, A. S., Fernandes-Alnemri, T., and Alnemri, E. S. (2002) J Biol Chem 277, 432-438
9. Verhagen, A. M., Silke, J., Ekert, P. G., Pakusch, M., Kaufmann, H., Connolly, L. M., Day, C. L., Tikoo, A., Burke, R., Wrobel, C., Moritz, R. L., Simpson, R. J., and Vaux, D. L. (2002) J Biol Chem 277, 445-454
10. Egger, L., Schneider, J., Rheme, C., Tapemoux, M., Hacki, J., and Borner, C. (2003) Cell Death Differ 10, 1188-1203
11. Martins, L. M., Turk, B. E., Cowling, V., Borg, A., Jarrell, E. T., Cantley, L. C., and Downward, J. (2003) J Biol Chem 278, 49417-49427
12. Okada, M., Adachi, S., Imai, T., Watanabe, K. I., Toyokuni, S. Y., Ueno, M., Zervos, A. S., Kroemer, G., and Nakahata, T. (2004) Blood 103(6), 2299-307
13. Jones, J. M., Datta, P., Srinivasula, S. M., Ji, W., Gupta, S., Zhang, Z., Davies, E., Hajnoczky, G., Saunders, T. L., Van Keuren, M. L., Fernandes-Alnemri, T., Meisler, M. H., and Alnemri, E. S. (2003) Nature 425, 721-727
14. Yang, Q. H., Church-Hajduk, R., Ren, J., Newton, M. L., and Du, C. (2003) Genes Dev 17, 1487-1496
15. Srinivasula, S. M., Gupta, S., Datta, P., Zhang, Z., Hegde, R., Cheong, N., Fernandes-Alnemri, T., and Alnemri, E. S. (2003) J Biol Chem 278, 31469-31472
16. L1, W., Srinivasula, S. M., Chai, J., Li, P., Wu, J. W., Zhang, Z., Alnemri, E. S., and Shi, Y. (2002) Nat Struct Biol 9, 436-441
17. Weiss, G. A., Watanabe, C. K., Zhong, A., Goddard, A., and Sidhu, S. S. (2000) Proc Natl Acad Sci USA 97, 8950-8954
18. Fuh, G., Pisabarro, M. T., L1, Y., Quan, C., Lasky, L. A., and Sidhu, S. S. (2000) J Biol Chem 275, 21486-21491
19. Sidhu, S. S., Lowman, H. B., Cunningham, B. C., and Wells, J. A. (2000) Methods Enzymol 328, 333-363
20. Skelton, N. J., Koehler, M. F., Zobel, K., Wong, W. L., Yeh, S., Pisabarro, M. T., Yin, J. P., Lasky, L. A., and Sidhu, S. S. (2003) J Biol Chem 278, 7645-7654
21. Kunkel, T. A., Roberts, J. D., and Zakour, R. A. (1987) Methods Enzymol 154, 367-382
22. Vajdos, F. F., Adams, C. W., Breece, T. N., Presta, L. G., de Vos, A. M., and Sidhu, S. S. (2002) J Mol Biol 320, 415-428
23. Daniels, D. L., Cohen, A. R., Anderson, J. M., and Brunger, A. T. (1998) Nat Struct Biol 5, 317-325
24. Doyle, D. A., Lee, A., Lewis, J., Kim, E., Sheng, M., and MacKinnon, R. (1996) Cell 85, 1067-1076
25. Songyang, Z., Fanning, A. S., Fu, C., Xu, J., Marfatia, S. M., Chishti, A. H., Crompton, A., Chan, A. C., Anderson, J. M., and Cantley, L. C. (1997) Science 275, 73-77
26. Schultz, J., Hoffmuller, U., Krause, G., Ashurst, J., Macias, M. J., Schmieder, P., Schneider-Mergener, J., and Oschkinat, H. (1998) Nat Struct Biol 5, 19-24
27. Karthikeyan, S., Leung, T., and Ladias, J. A. (2001) J Biol Chem 276, 19683-19686
28. Im, Y. J., Lee, J. H., Park, S. H., Park, S. J., Rho, S. H., Kang, G. B., Kim, E., and Eom, S. H. (2003) J Biol Chem 278, 48099-48104
29. Stricker, N. L., Christopherson, K. S., Yi, B. A., Schatz, P. J., Raab, R. W., Dawes, G., Bassett, D. E., Jr., Bredt, D. S., and Li, M. (1997) Nat Biotechnol 15, 336-342
30. Wang, S., Raab, R. W., Schatz, P. J., Guggino, W. B., and Li, M. (1998) FEBS Lett 427, 103-108
31. Feng, W., Fan, J. S., Jiang, M., Shi, Y. W., and Zhang, M. (2002) J Biol Chem 277, 41140-41146
32. Vaux, D. L., and Silke, J. (2003) Cell 115, 251-253

1. An isolated polypeptide that binds specifically to Omi PDZ, wherein said polypeptide comprises a sequence having two hydrophobic moieties separated by 1 or 2 amino acid positions, wherein the polypeptide does not comprise the sequence GQYYFV, GGIRRV or MDIELVMI.

2. The isolated polypeptide of claim 1, wherein at least one of the hydrophobic moieties is in the C-terminal region of the polypeptide.

3. The isolated polypeptide of claim 1, wherein one of the hydrophobic moieties comprises the carboxyl terminal amino acid residue of the polypeptide.

4. The isolated polypeptide of claim 1, wherein the carboxyl terminal amino acid residue is carboxylated.

5. The isolated polypeptide of claim 1, wherein one moiety is composed of about 2-4 hydrophobic clusters with aromatic residues in at least two amino acid positions and the other moiety is composed of about 1-2 hydrophobic amino acids with bulky side chain.

6. The isolated polypeptide of claim 5, wherein the amino acid with bulky side chain is Trp, Phe, Leu or Ile.

7. The isolated polypeptide of claim 1, wherein amino acid position −1 is W, wherein amino acid numbering is based on the C-terminus residue being in position 0.

8. The isolated polypeptide of claim 1, wherein position −2 is F, wherein amino acid numbering is based on the C-terminus residue being in position 0.

9. The isolated polypeptide of claim 1, wherein position −3 is M, wherein amino acid numbering is based on the C-terminus residue being in position 0.

10. The isolated polypeptide of claim 1, wherein a first hydrophobic moiety comprises the amino acids FWV, wherein F is in position −2, W in position −1 and V in position 0, and wherein position 0 is the C-terminal residue.

12. The isolated polypeptide of claim 1, wherein a second hydrophobic moiety comprises T in position −4.

13. The isolated polypeptide of claim 1, wherein a second hydrophobic moiety comprises F in position −5.

14. The isolated polypeptide of claim 1, wherein the sequence having the two hydrophobic moieties has the formula X1-H1-X2-X3-H2-X4-X5, wherein H1 and H2 are a first and second hydrophobic moiety respectively.

15. An isolated polypeptide that binds specifically to Omi PDZ and comprises either a carboxyl terminal, N-terminal or internal amino acid sequence having the sequence of a member selected from the group consisting of the sequences of Tables II and III.

16. The polypeptide of claim 15, wherein the carboxyl terminal amino acid sequence has the sequence WTMFWV.

17. The polypeptide of claim 15, wherein the carboxyl terminal amino acid sequence has the sequence RFPHFWV.

18. An isolated polypeptide comprising an amino acid sequence that competes with the polypeptide of any of claims 15-17 for binding to Omi PDZ sequence, wherein the polypeptide does not comprise the sequence GQYYFV, GGIRRV or MDIELVMI.
